(19) United States
(12) Patent Application Publication
(10) Pub. No.: US 2023/0279342 A1
(43) Pub. Date: Sep. 7, 2023

(54) METHOD FOR MEDIUM TREATMENT BEFORE INOCULATION

(71) Applicants: Hoffmann-La Roche Inc., Little Falls, NJ (US); Genentech, Inc., South San Francisco, CA (US)

(72) Inventors: Oliver Popp, Penzberg (DE); Masaru Ken Shiratori, Burlingame, CA (US)

(73) Assignees: Hoffmann-La Roche Inc., Little Falls, NJ (US); Genentech, Inc., South San Francisco, CA (US)

(21) Appl. No.: 17/964,325

(22) Filed: Oct. 12, 2022

Related U.S. Application Data
(63) Continuation of application No. PCT/EP2021/059690, filed on Apr. 14, 2021.
(60) Provisional application No. 63/010,536, filed on Apr. 15, 2020.

(51) Int. Cl. C12N 5/00; C12N 5/071

(52) U.S. Cl. CPC C12N 5/0018; C12N 5/0602; C12N 2500/20; C12N 2500/32; C12N 2523/00

(57) ABSTRACT

The disclosure relates to methods for handling and supplementation of cell culture medium to improve process performance in eukaryotic recombinant expression systems.

### CROSS-REFERENCE TO RELATED APPLICATIONS

This application is a continuation of International Application No. PCT/EP2021/059690 having an International filing date of Apr. 14, 2021, which claims benefit of priority to U.S. Provisional Application No. 63/010,536, filed on Apr. 15, 2020, the contents of which are incorporated herein by reference in their entirety.

### FIELD OF INVENTION

The disclosure relates to methods for handling and supplementation of cell culture medium to improve process performance in eukaryotic recombinant expression systems.

### BACKGROUND

There is an on-going demand for greater quantities of therapeutic recombinant proteins and thus consistent increases in protein production, cell growth and viability are sought via implementation of new methods to improve cell development, media optimisation and process control parameters.

L-cysteine is a key amino acid for cell growth, maintenance and protein production. It plays an essential role as a source of sulphur and is also the limiting factor for the synthesis of glutathione (GSH), the main intracellular antioxidant, as well as being required for the structure and folding of proteins, via disulphide and trisulphide bonds. L-cysteine has very low stability at a neutral pH and can thus cause media formulations to destabilise in the neutral conditions (about pH 6.5 to about pH 7.5) necessary for mammalian cell growth. Thus L-cysteine is often added in fed-batch fermentation processes in its disulphide form, cystine, which has a very low solubility at neutral pH. Hecklau et al., J. Biotech 218 (2016) 53-63 demonstrated that in batch experiments, cell proliferation was completely inhibited in cysteine depleted media but that supplementation with a cysteine derivative, S-sulphocysteine (SSC), which is stable in both media and feeds, can effectively replace L-cysteine and increase the duration of cell culture and cell titre. SSC was also discovered to reduce the trisulphide bond formation in proteins, which is of particular value in the production of antibodies.

Nishiuch et al., In Vitro 12(9) (1976) 635-638 discovered (a) that 1 mM cysteine was highly toxic to cultured cells when added to Eagle's Minimum Essential Medium supplemented with 10% bovine serum (MEM-10BS) and that 1.5 mM cysteine was similarly toxic when present as an original ingredient in CMRL 1066 supplemented with 10% bovine serum (CMRL-10BS); and (b) that when the cysteine containing media MEM-10BS and CMRL-10BS were incubated without cells for one day, the cysteine concentrations decreased. Medium pre-incubation reduced cysteine induced cytotoxicity.

Kuschelewski et al., Biotechnol. Prog., 33(3) (2017) 759-770 considered the effect of reactive species generated by cell culture media on the overall stability of the media and the behaviour of cells cultured in vitro. L-cysteine and other thiol-containing components of cell culture media are likely to be oxidised, forming H₂O₂ or free radical intermediates such as cysteinyl radicals, which can themselves form disulphides in the presence of metal catalysts such as copper or react with oxygen to form cys-SO₂H or react with H₂O₂ to form cys-SO₃H. The generation of oxidation products contributes to an increased cellular stress level.

There is a continuing need to optimise medium formulations and process parameters to improve cell viability, specific productivity and titre. Improvements to medium formulations and/or feeding strategies to result in desirable recombinant protein expression, titre, cell growth and/or cell viability can reduce the costs associated with manufacturing protein therapeutics.

### SUMMARY OF THE INVENTION

The method of the present disclosure entails implementing a conditioning period for a medium prior to inoculation with a recombinant protein-producing eukaryotic cell. Eukaryotic cells grown in the resulting medium show increased cell-specific productivity. Furthermore, hydrolytic activity and process specific oxygen demand is improved. Also, the recombinant protein generated by the cell cultured in the resulting medium comprises a decreased fraction of host cell protein impurities.

### FIGURES

FIG. 1: Cell specific productivity (qP) from Example 1 for clone 2. For each illustration, the data for the DoE cases “low TES, low CysCys” (A), “low TES, high CysCys” (B), and “high TES, high CysCys” (C) were normalized to the supplementation time “−0 h”.

FIG. 2: CHO derived host cell protein (CHO HCP) concentration from Example 1 for clone 2. For each illustration, the data for the DoE cases “low TES, low CysCys” (A), “low TES, high CysCys” (B), and “high TES, high CysCys” (C) were normalized to the supplementation time “−0 h”.

FIG. 3: Schematic illustration of the experimental setup performed in Example 2 (“Scenario 1”). In a method according to the invention, trace metals and cystine were added to the cell culture medium at the start of conditioning and incubated for 24 hours under conditions for inoculation in the absence of cells (in Example 1, referred to as “Aged”). As a comparison, cell culture medium was added to the bioreactor and was incubated under the same conditions for 24 hours, and trace metals and cystine were subsequently added to the cell culture medium directly before inoculation with cells (in Example 2, referred to as “Fresh”).

FIG. 4A: Viable cell density as assessed in Example 2 for clone 1.

FIG. 4B: Viable cell density as assessed in Example 2 for clone 2.

FIG. 4C: Viable cell density as assessed in Example 2 for clone 3.

FIG. 4D: Viable cell density as assessed in Example 2 for clone 4.

FIG. 5A: Product titer (global volumetric titer) as assessed in Example 2 for clone 1.

FIG. 5B: Product titer (global volumetric titer) as assessed in Example 2 for clone 2.

FIG. 5C: Product titer (global volumetric titer) as assessed in Example 2 for clone 3.

FIG. 6A: Cell specific productivity represented by the slope of cell-time integral vs titre as assessed in Example 2 for clone 1.

FIG. 6B: Biomass-specific productivity represented by the slope of cell-volume integral vs titre as assessed in Example 2 for clone 1.

FIG. 6C: Cell specific productivity represented by the slope of cell-time integral vs titre as assessed in Example 2 for clone 2.

FIG. 6D: Biomass-specific productivity represented by the slope of cell-volume integral vs titre as assessed in Example 2 for clone 2.

FIG. 6E: Cell specific productivity represented by the slope of cell-time integral vs titre as assessed in Example 2 for clone 3.

FIG. 6F: Biomass-specific productivity represented by the slope of cell-volume integral vs titre as assessed in Example 2 for clone 3.

FIG. 7A: Absolute cell-specific productivity as assessed in Example 2 for clones 1,2 and 3.

FIG. 7B: Relative cell-specific productivity, normalised to productivity of the indicated clones when medium is conditioned according to the invention (referred to as “Aged” in Example 2) as assessed in Example 2 for clones 1, 2 and 3.

FIG. 8A: Oxygen demand during fermentation as assessed in Example 2 for clone 1.

FIG. 8B: Oxygen demand during fermentation as assessed in Example 2 for clone 2.

FIG. 8C: Oxygen demand during fermentation as assessed in Example 2 for clone 3.

FIG. 8D: Oxygen demand during fermentation as assessed in Example 2 for a host cell line.

FIG. 8E: Absolute integral of oxygen demand as assessed in Example 2 for clones 1, 2, 3 and a host cell line.

FIG. 8F: Relative integral of oxygen demand normalised to productivity of the indicated clones when medium is conditioned according to the comparative setting (referred to as “Fresh” in Example 2) as assessed in Example 2 for clones 1, 2, 3 and a host cell line.

FIG. 9A: LDH (lactate dehydrogenase) activity in the fermentation supernatant as assessed in Example 2 for clones 1, 2, 3 and a host cell line.

FIG. 9B: LDH (lactate dehydrogenase) activity in the fermentation supernatant normalised to LDH activity of the indicated clones when medium is conditioned according to the comparative setting (referred to as “Fresh” in Example 2) as assessed in Example 2 for clones 1, 2, 3 and a host cell line.

FIG. 10A: Host cell protein (HCP) concentration in cell culture as assessed in Example 2 for clones 1, 2, 3 and a host cell line.

FIG. 10B: Normalised host cell protein (HCP) concentration in cell culture, normalised to productivity of the indicated clones when medium is conditioned according to the comparative setting (referred to as “Fresh” in Example 2) as assessed in Example 2 for clones 1, 2, 3 and a host cell line.

FIG. 11A: PLBL2 (Phospholipase B-like 2 protein) concentration in cell culture as assessed in Example 1 for clones 1, 2, 3 and a host cell line.

FIG. 11B: Normalised PLBL2 (Phospholipase B-like 2 protein) concentration in cell culture normalised to productivity of the indicated clones when medium is conditioned according to the comparative setting (referred to as “Fresh” in Example 2) as assessed in Example 2 for clones 1, 2, 3 and a host cell line.

FIG. 12A: Quality of produced antibody as measured by CE-SDS in Example 2 for clones 1, 2 and 3 at day 14 post inoculation.

FIG. 12B: Quality of produced antibody as measured by SEC monomer content as assessed in Example 2 for clones 1, 2 and 3 at day 14 post inoculation.

FIG. 13: Schematic illustration of the experimental setup performed in Example 3 (“Scenario 2”). In a method according to the invention, wherein the medium was held at inoculating conditions for 72 hours in the absence of cells, trace metals and cystine were added to the cell culture medium either at the start of conditioning (“−72 h”), 24 hours after the start of conditioning (“−48 h”) or 48 hours after the start of conditioning (“−24 h”).

FIG. 14: Product titre (global volumetric titre) as assessed in Example 3 for clone 2, wherein medium was conditioned according to the scenario indicated in FIG. 13.

FIG. 15: Absolute cell specific productivity as assessed in Example 3 for clone 2, where medium was conditioned according to the scenario indicated in FIG. 13. Error bars are relative standard deviations calculated from test case “−48 h” (n=3).

FIG. 16: Absolute oxygen mass flow in fermenter in-gas as assessed in Example 3 for clone 2, where medium was conditioned according to the scenario indicated in FIG. 13.

FIG. 17: Absolute integral of oxygen demand as assessed in Example 3 for clone 2, wherein medium was conditioned according to the scenario indicated in FIG. 13. Error bars are relative standard deviations calculated from test case “−48 h” (n=3):

FIG. 18: Schematic Diagram of combinations of “Fresh” (striped) and “Aged” (dotted) supplemented media beneficial for use in a cell fermentation process of the disclosure.

FIG. 19: Viable cell density values for setups with media supplemented with cystine and redox-active trace metals 0 hours before inoculation (“Fresh”) or supplementation 12, 24 or 48 hours prior to inoculation (“Aged”). “Fresh” supplemented media is beneficial for cell growth.

FIG. 20: Cell specific productivity shown for different supplementation and inoculation scenarios. “Aged” media is beneficial for cell-specific productivity.

FIG. 21: Titers measured for different supplementation and inoculation scenarios. Supplementation timing of cystine and redox-active trace metals does not affect product titer.

FIG. 22: CE-SDS main peak measurements (in %) for different supplementation and inoculation scenarios. Supplementation timing of cystine and redox-active trace metals does not affect CE-SDS product species abundance.

FIG. 23: SEC Monomer content (in %) for different supplementation and inoculation scenarios. Supplementation timing of cystine and redox-active trace metals does not affect SEC product species abundance.

FIG. 24: Host cell Protein content of cell culture supernatant for different supplementation and inoculation scenarios. Whiskers represent the standard deviation of three technical replicates (n=3)

FIG. 25: Clusterin content in cell culture supernatant for different supplementation and inoculation scenarios.

FIG. 26: PLBL2 content in cell culture supernatant for different supplementation and inoculation scenarios. Whiskers represent the standard deviation of three technical replicates (n=3)

FIG. 27: Oxygen demand for setups with media supplemented with cystine and redox-active trace metals 0 hours (“Fresh”), or 48 or 96 hours (“Aged”) before inoculation. Cell culture processes with “Fresh” supplemented media are less efficient in respect of oxygen demand.

### DESCRIPTION OF THE INVENTION

While the terminology used in this application is standard within the art, definitions of certain terms are provided herein to assure clarity and definiteness to the meaning of the claims. Units; prefixes, and symbols may be denoted in their SI accepted form. Numeric ranges recited herein are inclusive of the numbers defining the range and include and are supportive of each integer within the defined range. Unless otherwise noted, the terms “a” or an are to be construed as meaning “at least one of. The section headings used herein are for organizational purposes only and are not to be construed as limiting the subject matter described. The methods and techniques described herein are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). All documents, or portions of documents, cited in this application, including but not limited to patents, patent applications, articles, books, and treatises, are hereby expressly incorporated by reference.

The invention flows from the discovery that the timing of the supplementation of a medium with specific nutrients and the maintenance of the supplemented medium under conditions suitable for inoculation for a defined period before inoculation has advantageous results on cell-specific productivity, host cell protein (HCP) levels and content and process specific oxygen demand. Medium supplementation may take place before and/or during the period of maintenance of the medium under conditions suitable for inoculation.

Thus, the invention provides a method for treatment of a cell culture medium, wherein the method comprises: holding a cell culture medium in a vessel under conditions suitable for inoculation of the medium with a eukaryotic cell that has been engineered to recombinantly express an exogenous protein, characterised in that the medium comprises the following nutrients:
(i) one or more of cystine, cysteine and a cysteine derivative; and
(ii) one or more redox active trace metals;
and in that the medium comprising the nutrients is held under said conditions suitable for inoculation for a period of at least 10 hours in the absence of cells prior to inoculation.

In a further aspect, the present disclosure includes a method for production of a recombinant protein, the method comprising inoculating a conditioned cell culture medium with a eukaryotic cell engineered to recombinantly express an exogenous protein, wherein the medium is conditioned by holding the cell culture medium in a vessel under conditions suitable for inoculation of the medium with a eukaryotic cell that has been engineered to recombinantly express an exogenous protein, the medium comprising the following nutrients:
(i) one or more of cystine, cysteine and a cysteine derivative; and
(ii) one or more redox active trace metals;
wherein the medium comprising the nutrients is held under said conditions suitable for inoculation for a period of at least 10 hours in the absence of cells prior to inoculation thereof.

In the methods of the invention, a cell culture medium (also referred to herein simply as a medium) is added to a vessel, and the vessel is adjusted such that the conditions in the vessel are suitable for inoculation of the medium with a eukaryotic cell for production of a recombinant protein. As used herein a “recombinant protein” is considered to be the same as “an exogenous protein expressed recombinantly”. Further description of conditions suitable for inoculation are provided below.

A medium that has been supplemented and has been maintained in a vessel under conditions suitable for inoculation for a period of time, as defined above, is referred to herein as a “conditioned” medium. A “pre-inoculated” medium, as used herein, refers to the cell culture medium before inoculation with cells. In consequence, a pre-inoculated medium is cell-free. A pre-inoculated medium can be a conditioned medium. The “conditioning period” is the period during which the medium is held in the vessel under conditions suitable for inoculation in the absence of cells.

The conditioning period ends with inoculation of the medium. Thus, a time point referred to as “prior to inoculation” is included within the conditioning period.

A culture medium for growing a eukaryotic cell for expression of a recombinant protein comprises specific nutrients. The addition of nutrients to a medium is referred to as “supplementation”. Supplementation may be carried out during the conditioning period and/or before or at the start of the conditioning period. Prior to supplementation with specific nutrients the medium may be referred to herein as the “base” medium. The base medium is thus the medium to which all of the specific nutrients disclosed herein have not yet been added in their final concentration. In one embodiment, at least one of the nutrients (either the total amount, or a portion of the total amount to be added to the medium for medium supplementation) is added to the base medium during the conditioning period and at least 10 hours before the end thereof.

By “inoculation”, the present disclosure intends the act of introducing a eukaryotic cell or suspension of eukaryotic cells, which cells are engineered to recombinantly express an exogenous protein, into the medium in a vessel.

In the methods of the invention, the medium which is supplemented with the specific nutrients, i.e. one or more of cystine, cysteine and a cysteine derivative; and one or more redox active trace metals, is held in a vessel under conditions suitable for inoculation for a period of at least about 10 hours.

The inventors have discovered that holding the supplemented medium in a vessel before inoculation under conditions suitable for inoculation as realised by the invention, is beneficial to cell-specific productivity, host cell protein (HCP) content and process specific oxygen demand.

In one embodiment of the invention, the medium which is supplemented with the specific nutrients is held in a vessel under conditions suitable for inoculation for a period of at most about 96 hours. Said period can be for periods of whole hours, or can include a time period defined by minutes, or seconds. When a range is given for said period, the range includes both end points and every whole hour, minute or second falling within that range. In one embodiment of the invention the supplemented medium is held in the vessel for at least about 12 hours, at least about 14 hours, at least about 16 hours, at least about 18 hours, at least about 20 hours, at least about 22 hours, at least about 24 hours, at least about 48 hours, at least about 72 hours or at least about 96 hours. In one embodiment of the invention, the supplemented medium is held in the vessel for at least about 24 hours. In one embodiment, the supplemented medium is held in the vessel for from about 10 hours to about 96 hours. In one embodiment, the supplemented medium is held in the vessel for from about 24 hours to about 96 hours.

The method of the invention comprises supplementing the medium with one or more nutrients selected from the group consisting of: (1) one or more of cystine, cysteine and a cysteine derivative; and (2) one or more redox active trace metals. These may be referred to herein as “the nutrients” or “the specific nutrients”.

The nutrients may be, independently from each other, added to the medium in one dose or in divided doses. In one embodiment one of the nutrients is added to the medium in one dose. In one embodiment, all of the nutrients are added to the medium in one dose.

The nutrients may be added to the medium before or after addition of the medium to the vessel. The nutrients may be added to the medium before or after inoculation conditions are attained within the vessel. Thus, the medium, supplemented or not, may be added to the vessel and then inoculation conditions implemented and attained. Alternatively, inoculation conditions may be implemented in the vessel before addition of the medium (supplemented or not), and then those conditions maintained.

The nutrients may be added during medium preparation and the medium may then be stored under typical storage conditions (e.g. at 4° C.) for up to 3 months.

In one embodiment of the invention one or more of cystine, cysteine and a cysteine derivative is added to the medium before addition of the medium to the vessel. In one embodiment of the invention one or more of cystine, cysteine and a cysteine derivative is added to the medium in the vessel before inoculation conditions are attained within the vessel. In one embodiment of the invention one or more of cystine, cysteine and a cysteine derivative is added to the medium in the vessel after inoculation conditions are attained within the vessel.

In one embodiment of the invention one or more redox active trace metals is added to the medium before addition of the medium to the vessel. In one embodiment of the invention one or more redox active trace metals is added to the medium in the vessel before inoculation conditions are attained within the vessel. In one embodiment of the invention one or more redox active trace metals is added to the medium in the vessel after inoculation conditions are attained within the vessel.

According to the method of the invention, the medium comprises one or more of cystine, cysteine and a cysteine derivative. Cystine is the oxidised dimer form of cysteine and is interconvertible with cysteine. The sulphur atoms bound to these amino acids provide sites for redox activity and electron transfer. Cysteine is also referred to as L-cysteine.

In one embodiment, the cysteine derivative is selected from the group of S-sulfocysteine, S-sulfocysteinylglycine, N-acetyl cysteine (NAC), cysteine S-linked N-acetyl glucosamine (GIcNAC-cys), homocysteine, L-cysteine mixed disulphides or L-cysteine mixed peptides, S-alkylated cysteine or cysteine with a thiol-protecting group, e.g FMOC-protected cysteine, reduced and oxidized glutathione (GSH), S-sulfoglutathione, γ-glutamylcysteine, cysteinylglycine, N-butanoyl α-glutamyl-cysteinyl-glycine, S-acyl-GSH and S-carboxy-L-cysteine.

In one embodiment, the cysteine derivative is selected from the group of S-sulfocysteine, N-acetyl cysteine (NAC), homocysteine and reduced and oxidized glutathione (GSH).

In one embodiment, the medium is supplemented with cystine. In one embodiment the medium is supplemented with cysteine. In one embodiment the medium is supplemented with a cysteine derivative. In one embodiment, the medium is supplemented with a mixture of cystine and cysteine, cystine and a cysteine derivative or cysteine and a cysteine derivative. In one embodiment, the medium is supplemented with a mixture of cystine, cysteine and a cysteine derivative.

In one embodiment of the invention, the medium comprises (after supplementation) one or more of cystine, cysteine and a cysteine derivative in a total concentration of about 0.5 mM to about 16 mM cysteine when held in the vessel under conditions suitable for inoculation. In one embodiment, the medium comprises about 1 mM to about 10 mM of one or more of cystine, cysteine and a cysteine derivative. In one embodiment, the medium comprises about 2 mM to about 8 mM of one or more of cystine, cysteine and a cysteine derivative.

In one embodiment, the method comprises the addition of cystine to the medium, with the cystine being added in an amount of about 0.25 mM to about 8 mM, in one embodiment about 0.5 mM to about 5 mM and in one embodiment about 1 mM to about 4 mM cystine.

In one embodiment, the method comprises the addition of cystine to the medium in the vessel at the start of the conditioning period.

In one embodiment, the method comprises the addition of cystine to the medium immediately prior to or at the same time as addition of the medium to the vessel before or at the start of the conditioning period.

In one embodiment, the method comprises the addition of cystine to the medium in the vessel, wherein the cystine is added as a single dose to the medium in the vessel at the start of the conditioning period and the supplemented medium is held in the vessel at conditions suitable for inoculation for at least about 24 hours prior to inoculation of the medium.

In one embodiment, the method comprises the addition of cystine to the medium as a single dose immediately prior to or at the same time as addition of the medium to the vessel, and the supplemented medium is held in the vessel at conditions suitable for inoculation for at least about 24 hours prior to inoculation of the medium. In one embodiment, conditions in the vessel are suitable for inoculation before addition of the (supplemented) medium thereto. In one embodiment, conditions suitable for inoculation are attained in the vessel after addition of the (supplemented) medium thereto.

In one embodiment of the invention, the one or more of cystine, cysteine and a cysteine derivative is added to the medium in solid form. In one embodiment of the invention, the one or more of cystine, cysteine and a cysteine derivative is added to the medium in solution.

In one embodiment of the present invention the medium is supplemented with one or more of cystine, cysteine and a cysteine derivative:
(i) before addition of the medium to the vessel;
(ii) at the same time as addition of the medium to the vessel;
(iii) after addition of the medium to the vessel, i.e. in the vessel, and before the vessel/medium is under conditions suitable for inoculation of the medium with the eukaryotic cell; or
(iv) in the vessel, when the vessel and the medium contained therein are under conditions suitable for inoculation of the medium with the eukaryotic cell.

According to the method of the invention, the medium comprises one or more redox active trace metals.

Redox active trace metals may be any redox active trace metals appropriate for cell growth in culture and recombinant protein production. In one embodiment, the one or more redox active trace metals are selected from iron, copper, chromium, cobalt, selenium, manganese, vanadium and molybdenum.

In one embodiment, the redox active trace metal is added to the medium in the form of a redox active trace metal salt. In one embodiment, the redox active trace metal is added to the medium in the form of a solution of a redox active trace metal salt. Any salt appropriate for inclusion in a culture medium for the production of a recombinant protein can be used. In one embodiment, the redox active trace metal salt is in the form a metal sulphate, halide, oxide, nitrate, citrate, acetate or phosphate. In one embodiment, the redox active trace metal salt is in hydrated or anhydrous form. In one embodiment, the metal ion is bound to a chelator such as transferrin or lactoferrin.

In one embodiment, iron salts appropriate for use in the present method are in the form of an iron sulphate, halide, oxide, nitrate, citrate, acetate or phosphate and are, for example, selected from Fe(III)-citrate, FeSO₄, FeCl₂, FeCl₃, Fe(NO₃)₃ and FePO₄ as well as iron bound to transferrin or lactoferrin.

In one embodiment, copper salts appropriate for use in the present method are in the form of a copper sulphate, halide, oxide, nitrate, citrate, acetate or phosphate and are, for example, selected from CuSO₄, CuCl₂, CuCO₃Cu(OH)₂, Cu(OH)₂, Cu(NO₃)₂, CuO and copper acetate such as Cu(C₂H₃O₂)₂2·H₂O.

In one embodiment, chromium salts appropriate for use in the present method are in the form of a chromium sulphate, halide, oxide, nitrate, citrate, acetate or phosphate and are, for example, selected from CrCl₂, CrCl₃, CrCl₃·6H₂O, C₁₂H₂₄Cl₃CRO₃, CrF₃·4H₂O, Cr(NO₃)₃·9H₂O, Cr₂O₃, Cr₂(SO₄)₃·xH₂O, CrO₂Cl₂, and K₃Cr(C₂O₄)₃·3H₂O.

In one embodiment, cobalt salts appropriate for use in the present method are in the form of a cobalt sulphate, halide, oxide, nitrate, citrate, acetate or phosphate and are, for example, selected from CoCl₂, CoCl₂·6H₂O, Co(NO₃)₂, Co(ClO₄)₂·6H₂O, Co(NO₃)₂·6H₂O, and CoSO₄·7H₂O.

In one embodiment, selenium salts appropriate for use in the present method are in the form of a selenium sulphate, halide, oxide, nitrate, citrate, acetate or phosphate and are, for example, selected from Na₂SeO₃, Na₂SeO₄, H₂SeO₄, H₂SeO₃, SeCl₄, and SeOCl₂.

In one embodiment, manganese salts appropriate for use in the present method are in the form of a manganese sulphate, halide, oxide, nitrate, citrate, acetate or phosphate and are, for example, selected from MnSO₄, MnCl₂, MnF₂ and MnI₂.

In one embodiment, vanadium salts appropriate for use in the present method are in the form of a vanadium sulphate, halide, oxide, nitrate, citrate, acetate or phosphate and are, for example, selected from VCl₂, VCl₃, VOCl₃, Na₃VO₄, NH₄VO₃, VOSO₄, and NaVO₃.

In one embodiment, molybdenum salts appropriate for use in the present method are in the form a molybdenum sulphate, halide, oxide, nitrate, citrate, acetate or phosphate and are, for example, selected from (NH₄)₆Mo₇O₂₄·4H₂O, H₃PMo₁₂O₄₀, and MoO₃·H₂O.

The redox active trace metals are typically used in solution.

The amounts of redox active trace metals required by cells for growth and protein production in culture are known in the art and the amounts of these redox active trace metals added to the medium before and/or during the conditioning period will follow those norms. In certain cases, e.g. when a base medium already contains an amount of a trace metal selected for addition according to the method of this disclosure, then the amount of redox active trace metal added can be adapted such that the total amount of redox active trace metal in the medium at the time of inoculation is that standard in the art. Further information can be gained from Yuk et al., (2014) Biotechnol Progress 31(1): 226-238 and Crowell et al., (2007) Biotechnol Bioeng 15, 96(3):538-549.

Redox active trace metals can be added to the medium in combinations, such as in Trace Element Solutions (TES). TES are formulated depending on the needs of the cells for redox active trace metals during growth and protein production. TES can comprise one or more, two or more, three or more, four or more, five or more, six or more, seven or more or all eight of the redox active trace metals described above. In one embodiment, one or more redox active trace metals is/are added individually to the medium and further redox active trace metals can be added to the medium as a TES.

In one embodiment, the medium comprises one or more of the redox active trace metals in the following concentrations: 1-200 μM iron, 0.01-5 μM copper, 0.01-100 nM chromium, 0.001-200 μM cobalt, 0.01-200 μM selenium, 0.01-5 μM manganese, 0.001-200 μM vanadium and/or 0.001-200 μM molybdenum, when held in the vessel under conditions suitable for inoculation. In one embodiment, the medium comprises one or more redox active trace metals in the following concentrations: 5-150 μM iron, 0.02-2 μM copper, 0.02-50 nM chromium, 0.005-150 μM cobalt, 0.02-150 μM selenium, 0.02-3 μM manganese, 0.005-150 μM vanadium/or and 0.005-150 μM molybdenum, when held in the vessel under conditions suitable for inoculation. In one embodiment, the medium comprises one or more redox active trace metals in the following concentrations: 10-100 μM iron, 0.05-1.5 μM copper, 0.1-30 nM chromium, 0.01-100 μM cobalt, 0.05-100 μM selenium, 0.1-3 μM manganese, 0.01-100 μM vanadium and/or 0.01-100 μM molybdenum, when held in the vessel under conditions suitable for inoculation.

In one embodiment of the present invention the medium is supplemented with one or more redox active trace metals:
(i) before addition of the medium to the vessel;
(ii) at the same time as addition of the medium to the vessel;
(iii) after addition of the medium to the vessel, i.e. in the vessel, and before the vessel is under conditions suitable for inoculation of the medium with the eukaryotic cell; or
(iv) in the vessel, when the vessel is under conditions suitable for inoculation of the medium with the eukaryotic cell.

In one embodiment of the invention, the medium is supplemented with cystine and iron and/or one or more, two or more, three or more, four or more, five or more, six or more or all of copper, chromium, cobalt, selenium, manganese, vanadium and molybdenum. In one embodiment, the medium is supplemented with cystine and iron. In one embodiment, the medium is supplemented with about 0.25 mM to about 8 mM cystine, in one embodiment supplementation is with about 0.5 mM to about 5 mM cystine and in one embodiment supplementation is with about 1 mM to about 4 mM cystine. In one embodiment, the medium is supplemented with about 1-200 μM iron, in one embodiment supplementation is with about 5 to about 150 μM iron and in one embodiment supplementation is with about 10 to about 100 μM iron.

If iron and/or one or more of copper, chromium, cobalt, selenium, manganese, vanadium and molybdenum are added to the medium as a TES, the concentration of the TES added to the medium will depend on the nature of the individual components and the concentration of each thereof. The effective redox active trace metal concentration C_{TES} is calculated according to the equation:

C_{TES}=□n_{trace elements}/V (Equ 1)

wherein TES is Trace Element Solution, n is amount of each trace metal in mol, V is TES volume in L.

In one embodiment, the amount of a TES added to the medium will be about 6 μM to about 350 μM. In one embodiment, the amount of a TES added to the medium will be about 10 μM to about 250 μM. In one embodiment, the amount of a TES added to the medium will be about 15 μM to about 100 μM.

TES are further described in the Examples.

In one aspect of the above embodiment, the supplemented medium is held in the vessel at inoculating conditions for at least 24 hours prior to inoculation.

In the methods of this disclosure inoculation of cells into a cell culture medium may take place more than once during the cell fermentation process. Typically cells may be inoculated into a cell culture medium for the seed train, the growth phase and for the production phase. In this disclosure the cell culture medium inoculated for one or both of the growth phase and the production phase is supplemented and conditioned as described herein. Biphasic process phases within a process step, e.g. within the growth phase and/or within the production phase, enable the use of either or both of a “Fresh” and “Aged” medium in that phase.

The methods of this disclosure enable the cell culture medium used in the fermentation process to be tailored, by conditioning as described herein, to benefit cell growth and cell specific productivity.

In one aspect of this embodiment, when inoculation is in respect of the growth phase, a cell culture medium therefor is held in a vessel under conditions suitable for inoculation of the medium with a eukaryotic cell that has been engineered to recombinantly express an exogenous protein, wherein the medium comprises:
one or more of cystine, cysteine and a cysteine derivative; and
one or more redox active trace metals and wherein the medium comprising the nutrients is held under said conditions suitable for inoculation for a period of at least 10 hours in the absence of cells prior to inoculation.

In one aspect of this embodiment, the growth phase can be multi-phasic, with the option for using differently treated media (i.e. “Aged” or “Fresh”) for different phases within the growth phase. Thus in this aspect one or more phases, or all phases, of the growth phase are in a medium conditioned according to this disclosure. In one aspect the medium for the N-1 phase is conditioned according to this disclosure.

In one aspect of this embodiment, when inoculation is in respect of the production phase, a cell culture medium therefor is held in a vessel under conditions suitable for inoculation of the medium with a eukaryotic cell that has been engineered to recombinantly express an exogenous protein, wherein the medium comprises:
one or more of cystine, cysteine and a cysteine derivative; and
one or more redox active trace metals and wherein the medium comprising the nutrients is held under said conditions suitable for inoculation for a period of at least 10 hours in the absence of cells prior to inoculation.

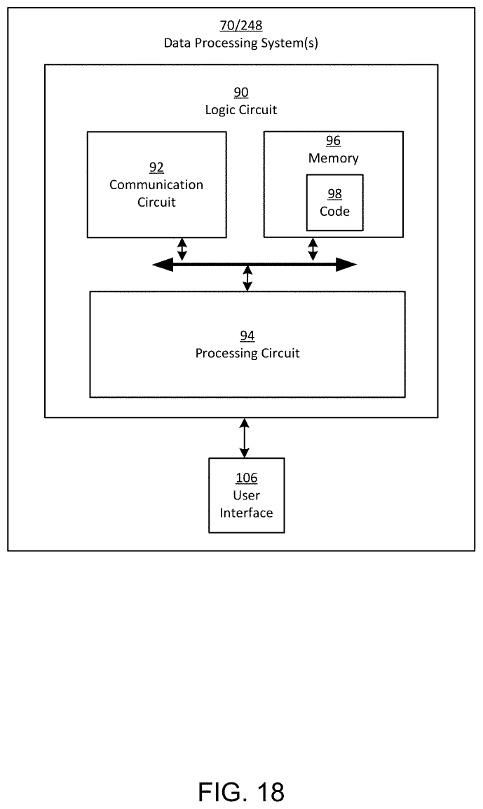

In one aspect of this embodiment, the production phase can be biphasic, such that one phase thereof is in a medium conditioned according to this disclosure and one phase thereof is in a medium not subject to conditioning according to this disclosure. FIG. 18 illustrates this aspect of the disclosure.

In one aspect of this embodiment, when inoculation is in respect of the growth phase and the production phase, a cell culture medium for the growth phase is held in a vessel and a cell culture medium for the production phase is held in a vessel, the vessels being held under conditions suitable for inoculation of the medium with a eukaryotic cell that has been engineered to recombinantly express an exogenous protein, wherein the medium comprises:
one or more of cystine, cysteine and a cysteine derivative; and
one or more redox active trace metals and wherein the medium comprising the nutrients is held under said conditions suitable for inoculation for a period of at least 10 hours in the absence of cells prior to inoculation.

In one aspect of this embodiment, it is preferred that an “Aged” medium is not used for both the N-1 and N phases of the cell fermentation process.

In these aspects, the time for which the medium is held under conditions suitable for inoculation in the absence of cells prior to inoculation may be the same or different for each of the different phases of the cell fermentation process.

It has been discovered that cell-specific productivity measured in the production phase of the cell fermentation process can be increased when the medium for the growth phase or the medium for the production phase is supplemented and conditioned before inoculation (“Aged”). In this aspect, it is preferred that, when the medium for the growth phase is supplemented and conditioned before inoculation (“Aged”), the medium for the production phase is “Fresh” and when the medium for the production phase is supplemented and conditioned before inoculation (“Aged”), the medium for the growth phase is “Fresh”.

The cell culture medium for use in the method of this disclosure is chemically defined, such that the components of the medium are known and controlled.

The cell culture medium should contain a balanced set of essential nutrients in a ratio that meets the demand of the cells for cell proliferation and production of the pharmaceutical protein. Cell culture media have been extensively developed and published in recent history, including such media for culture of eukaryotic cells. All components of defined media are well characterized and such media do not contain complex additives such as serum and hydrolysates. Typically, these media include defined quantities of purified growth factors, proteins, lipoproteins and other substances which may otherwise be provided by serum or extract supplement. Such media have been produced with the sole purpose of supporting highly productive cell cultures. Certain defined media may be termed low protein media or may be protein free if the typical components of low protein media, insulin and transferrin, are not included.

The medium for use in the method of this disclosure may also be serum free and thus will not contain any animal-derived components, fetal bovine serum, bovine serum albumin or human serum albumin.

Examples of commercially available culture media include Ham's F10 (Sigma), Minimal Essential Medium (MEM, Sigma), RPMI-1640 (Sigma) and Dulbecco's Modified Eagle's Medium (DMEM, Sigma). Chemically defined media designed for culture of CHO cells in particular include CD FortiCHO™ (Forti, or FortiCHO) Medium, CD OptiCHO™ (Opti, or OptiCHO) Medium, CD-CHO Medium (all available from Life Technologies) and ActiCHO-P (Acti, or ActiCHO) medium (available from GE Healthcare). Any such media may be supplemented as necessary with hormones and/or other growth factors (such as insulin, transferrin or epidermal growth factor); salts (such as sodium chloride, calcium, magnesium and phosphate), buffers (such as HEPES); nucleosides (such as adenosine and thymidine), amino acids, antibiotics (such as GENTAMYCIN™), and glucose or an equivalent energy source. The medium may further comprise a hydrogen peroxide scavenger. Exemplary hydrogen peroxide scavengers are keto acids, such as pyruvate, α-ketoglutarate, oxaloacetate, acetoacetic acid and levulinic acid.

The necessary nutrients and growth factors for the medium including their concentrations, for growth of a particular cell line, are determined empirically and without undue experimentation as described in, for example, Mammalian Cell Culture, Mather (Plenum Press: NY 1984); Barnes and Sato, Cell 22 (1980) 649 or Mammalian Cell Biotechnology: A Practical Approach M. Butler (IRL Press, 1991). A suitable medium contains a basal medium component, such as DMEM/HAM F12-based formulation with modified concentrations of some components, such as amino acids, salts, sugar and vitamins, and optionally containing glycine, hypoxanthine, thymidine, recombinant human insulin, hydrolyzed peptone, such as PRIMATONE HS™ or PRIMATONE RL™ (Sheffield, England) or the equivalent, a cell protective agent, such as PLURONIC F68™ or the equivalent pluronic polyol and GENTAMYCIN™. In addition a hydrogen peroxide scavenger such as pyruvate, α-ketoglutarate, oxaloacetate, acetoacetic acid or levulinic acid may be included in or supplemented to the base medium.

As set out above, the medium used in accordance with the invention comprises specific nutrients. As defined above, the base medium is the medium to which all of the specific nutrients disclosed herein have not yet been added in their final concentration. Thus, in one aspect, the base medium does not contain any or substantially any cystine, cysteine or cysteine derivative. In one aspect, the base medium does not contain any or substantially any redox active trace metals. In one aspect, the base medium does not contain any or substantially any cystine, cysteine or cysteine derivative or any or substantially any redox active trace metals.

In the method of the present invention, the medium in the vessel is held for the conditioning period under conditions suitable for inoculation with a eukaryotic cell. A “vessel” as used herein is any container in which medium can be held under conditions suitable for inoculation. A vessel may be a multi-use or a single-use container. A vessel may have rigid walls, semi-rigid walls or a combination thereof, or be constructed partly or entirely from a flexible material. In the present disclosure, “conditions suitable for inoculation” are used in respect of the vessel and/or the medium contained therein. At the minimum, a “vessel” should allow control of temperature and/or pH and/or gas. Typically culture vessels will provide a contamination barrier to protect the medium from the external environment while maintaining the proper internal environment. For anchorage-dependent cells, the vessels provide a suitable and consistent substrate for cell attachment. The vessel may be used to hold the medium for all or a part of the pre-inoculation (conditioning) period and/or post-inoculation culture. In one aspect, the medium is held in a first vessel for all or a part of the conditioning period and then transferred to a further vessel before (for a part of the conditioning period) or at the time of inoculation. In another aspect, the medium is held in the same vessel for all of the conditioning period and for medium inoculation. The nature of the vessel after inoculation is not important to the present invention, so long as it enables growth of the culture and production of the recombinant protein.

In one embodiment, the vessel is a bioreactor. As used herein a “bioreactor” is an in vitro culture system that has been designed to initiate, maintain and direct cell growth in a well-defined and tightly controlled culture environment. Typically bioreactors are constructed from e.g. stainless steel and are multi-use, i.e. are used, cleaned/sterilised and re-used. However, the methods of the present disclosure can also be performed in single-use (disposable or re-cyclable) bioreactors, such as bags constructed partly or entirely from plastics. Bioreactors simulate a natural biochemical environment for the optimum growth of cells or tissues in microbial and cell culture. Differing reactor volumes may be used through the fermentation process, with bioreactor types ranging from small, less than 1 L benchtop units to 10,000 L systems for large-scale industrial applications. In the present disclosure, the cell culture may be established by inoculating either shake flasks or a 20 L bioreactor and cultivating for about 21 days. After that, cells may be transferred to an 80 L bioreactor for about 3 days, a 400 L reactor for about 3 days and a 2,000 L reactor for about 2 days (stage n-1). The main fermentation, for production of antibody (n phase), may take place in, for example, a 12,000 L bioreactor. In one aspect, the medium is held in a first vessel for all or a part of the conditioning period and then transferred to a bioreactor before (for a part of the conditioning period) or at the time of inoculation. In another aspect, the medium is held in a bioreactor for all of the conditioning period and for medium inoculation.

Conditions suitable for inoculation with a eukaryotic cell are standard in the art. These may depend on the eukaryotic cell selected for protein expression. Reactors, temperatures and other conditions for inoculation and fermentation culture of cells for biomass generation and the production of recombinant proteins, such as oxygen concentration, carbon dioxide and pH, agitation, temperature and humidity are known in the art.

In some embodiments, with respect to the medium in the vessel, conditions suitable for inoculation with a eukaryotic cell engineered to recombinantly express an exogenous protein include: a pH of about pH 6.0 to about pH 8.0, a temperature of about 20° C. to about 39° C., and a % O₂ of about 10% to about 80%; for example a pH of about pH 6.5 to about pH 7.5, a temperature of about 30° C. to about 38° C., and a % O₂ of about 15% to about 70%; and in one case a pH of about pH 6.8 to about pH 7.2, a temperature of about 35° C. to about 38° C., and a % O₂ of about 30% to about 60%. Depending on the vessel used, the conditions suitable for inoculation may also include medium agitation, for example at rates of about 10 rpm to over 1000 rpm. Typically, depending on the size of the vessel, this would be provided by an rpm of about 100-150, or an rpm of about 110-140, or an rpm of about 120-130. Any combination of the above specifically recited ranges is also envisaged.

Means for measuring the pH, temperature and O₂ levels are well known in the art, together with means for balancing the pH during the conditioning period should this fall outside the above ranges. In one aspect, the pH may be balanced during the conditioning period by the addition of CO₂ or sodium carbonate, with the amount added being monitored until the desired pH is achieved.

In another aspect, the invention relates to a cell culture medium obtainable by the method of treatment of a cell culture medium according to the invention.

In a further aspect, the invention relates to the use of the medium obtained by a method for treatment of a cell culture medium according to the invention in a eukaryotic cell fermentation process for production of recombinant protein.

Another aspect of the invention is a method for improving cell specific productivity of a eukaryotic cell engineered to recombinantly express an exogenous protein, the method comprising:
subjecting a cell culture medium to a method for treatment of a cell culture medium according to the invention to obtain a treated cell culture medium, and
cultivating a eukaryotic cell engineered to recombinantly express an exogenous protein in the treated medium, wherein the eukaryotic cell has a higher cell specific productivity when compared to the cell specific productivity of the eukaryotic cell when cultured in an identical cell culture medium, to which the nutrients were supplemented directly prior to inoculation.

Another aspect of the invention is the use of a method for treatment of a cell culture medium according to the invention for improving cell specific productivity of a eukaryotic cell engineered to recombinantly express an exogenous protein.

Another aspect of the invention is a method for reducing oxygen demand of a eukaryotic cell fermentation process, the method comprising:
subjecting a cell culture medium to a method for treatment of a cell culture medium according to the invention to obtain a treated cell culture medium, and
cultivating a eukaryotic cell engineered to recombinantly express an exogenous protein in the treated medium, wherein the cultivation has a lower oxygen demand when compared to the oxygen demand of a cultivation in an identical cell culture medium, to which the nutrients were supplemented directly prior to inoculation.

Another aspect of the invention is the use of a method for treatment of a cell culture medium according to the invention for reducing oxygen demand of a eukaryotic cell fermentation process.

Another aspect of the invention is a method for reducing the concentration of Phospholipase B-like 2 protein (PLBL2) in a cell culture, the method comprising:
subjecting a cell culture medium to a method for treatment of a cell culture medium according to the invention to obtain a treated cell culture medium, and
cultivating a eukaryotic cell engineered to recombinantly express an exogenous protein in the treated medium, wherein during the cultivation a lower amount of PLBL2 is formed a compared to the amount of PLBL2 formed during a cultivation in an identical cell culture medium, to which the nutrients were supplemented directly prior to inoculation.

Another aspect of the invention is the use of a method for treatment of a cell culture medium according to the invention for reducing the amount of Phospholipase B-like 2 protein (PLBL2) in a cell culture.

A further aspect of the invention is a method for producing an exogenous protein by recombinant expression from a eukaryotic cell, the method comprising:
subjecting a cell culture medium suitable for cultivating said eukaryotic cell to the method for treatment of a cell culture medium according to the invention to obtain a treated cell culture medium,
inoculating the treated cell culture medium with a eukaryotic cell engineered to recombinantly express an exogenous protein to form a cell culture,
cultivating the cell culture so that the exogenous protein is produced.

Methods of cultivating eukaryotic cells to allow recombinant production of exogenous protein are known in the art. Also, methods for inoculating the medium and the amount of inoculum are standard in the art.

The present disclosure provides cell culture under fermentation culture conditions. This is typically a multi-step culture procedure where the cells are cultivated in a number of steps or phases in an appropriate culture vessel. According to this preferred procedure, the fermentation culture process, e.g. from fresh or frozen vials of cells, typically covers three distinct phases, i.e.:
i) the seed train, for recovery of the cells e.g. after the stress of thawing and to normalize cell doubling times, which can last between 3 and e.g. more than 60 days, depending on the speed of cell recovery and the scale of production. Direct thawing is also envisaged;
ii) the growth phase, or inoculation/inoculum train, called N-x phases (N is the production phase), wherein x is typically 1 to 5, preferably 1 or 2. These phases may also be referred to as a growth phase(s) wherein cells are inoculated into a medium suitable for promoting growth and biomass generation. Thus, the N-x phases are typically for the expansion of the culture for larger cultivation formats and the wash-out of the selected compound; and
iii) the production phase, or N-phase, for the production of the recombinant protein in appropriate quantity and/or quality. The duration of this phase may depend on, for example, the nature of the recombinant cell as well as the quantity and/or quality of the expressed glycoprotein.

The seed train is, in some cases, optional.

The medium used in the methods of the present invention is suitable for use in a eukaryotic cell fermentation process. Any eukaryotic cell susceptible to cell culture and to expression of recombinant protein may be used in accordance with the present invention. The eukaryotic cell is typically a eukaryotic cell line which is capable of growth and survival when placed in suspension culture in a medium containing the appropriate nutrients and growth factors and which is typically capable of expressing and secreting large quantities of a recombinant protein of interest into the culture medium.

In one embodiment, the eukaryotic cell is a mammalian cell, a yeast cell or an insect cell. In one embodiment, the eukaryotic cell is a mammalian cell.

In one embodiment the mammalian cell is selected from an NSO murine myeloma cell line, a monkey kidney CVI line transformed by SV40 (COS-7, ATCC® CRL 1651); human embryonic kidney line 293S (Graham et al., J. Gen. Virol. 36 (1977) 59); baby hamster kidney cells (BHK, ATCC® CCL 10); mouse sertoli cells (TM4, Mather, Biol. Reprod. 23 (1980) 243); monkey kidney cells (CVI-76, ATCC® CCL 70); African green monkey kidney cells (VERO-76, ATCC® CRL 1587); human cervical carcinoma cells (HELA, ATCC® CCL 2): canine kidney cells (MDCK, ATCC® CCL 34); buffalo rat liver cells (BRL 3A, ATCC® CRL 1442); human lung cells (W138, ATCC® CCL 75): human liver cells (Hep G2, HB 8065); mouse mammary tumour cells (MMT 060562, ATCC® CCL 51); rat hepatoma cells (HTC, MI.54, Baumann et al., J. Cell Biol., 85 (1980) 1); and TR-1 cells (Mather et al., Annals N.Y. Acad. Sci. 383 (1982) 44), the PER.C6 cell line (Percivia LLC), hybridoma cell lines, and Chinese Hamster Ovary cells (CHO, Urlaub and Chasin P.N.A.S. 77 (1980) 4216)

In one embodiment the mammalian cell is a CHO cell or a derivative thereof. In one embodiment the derivative of a CHO cell is selected from CHO/-DHFR (Urlab & Chasin, supra), CHOK1SV (Lonza), CHO-K1 DUC B11 (Simonsen and Levinson P.N.A.S. 80 (1983) 2495-2499) and DP12 CHO cells (EP 307,247).

In one embodiment the mammalian cell is a CHO cell. CHO cells have become the gold-standard mammalian host cells for the production of therapeutic antibodies and most of these cell lines have been adapted to grow in suspension culture and are well-suited for reactor culture, scale-up and large volume production.

In one embodiment, the eukaryotic cell is a yeast cell. In one embodiment, the yeast cell is Saccharomyces cerevisiae or Pichia pastoris.

In one embodiment the eukaryotic cell is an insect cell. In one embodiment the insect cell is Sf-9.

The eukaryotic cell used in the methods of this disclosure is selected or manipulated to produce recombinant protein, typically glycosylated monoclonal antibody. Methods for manipulating eukaryotic cells to recombinantly produce exogenous protein are known in the art. Manipulation includes one or more genetic modifications such as introduction of one or more heterologous genes encoding the protein to be expressed. The heterologous gene may encode a protein either that is normally expressed in that cell or that is foreign to the host cell. Manipulation may additionally or alternatively be to up- or down-regulate one or more endogenous genes. Often, cells are manipulated to produce recombinant protein by, for example, introduction of a gene encoding the protein and/or by introduction of control elements that regulate expression of the gene encoding the protein. Genes encoding protein and/or control elements may be introduced into the host cell via vectors, such as a plasmid, phage or viral vector. Certain vectors are capable or autonomous replication in a host cell into which they are introduced whilst other vectors can be integrated into the genome of a host cell and are thereby replicated along with the host genome. Various vectors are publicly available and the precise nature of the vectors is not essential to the present disclosure. Typically vector components include one or more of a signal sequence, an origin of replication, one or more marker genes, a promoter and a transcription termination sequence. Such components are as described in WO 97/25428.

A “protein” as used herein is a molecule comprising two or more amino acid residues joined to each other by peptide bonds. Proteins may comprise modifications including, but not limited to, glycosylation, lipid attachment, sulfation, gamma-carboxylation of glutamic acid residues, hydroxylation and ADP-ribosylation.

In one embodiment, the recombinant protein is selected from antibodies, enzymes, receptors and ligands, or fusion proteins comprising antibodies, receptors or ligands. In one embodiment the exogenous protein is an antibody or a fusion protein thereof. In one embodiment the exogenous protein is an antibody. In one embodiment the antibody is a therapeutic antibody. In one embodiment the antibody is a multispecific antibody. In one embodiment the antibody is of IgG isotype. In one embodiment the antibody is of IgG1 isotype. In one embodiment the antibody is a full length antibody. In one embodiment the antibody is an antibody fragment. In one embodiment the antibody is a humanized antibody or a human antibody.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

In one embodiment the recombinant protein is a receptor. In one embodiment, the recombinant protein is a ligand and in one embodiment a cytokine. Recombinant proteins are produced by cell culture methods known in the art and may also be referred to as “exogenous protein”.

The recombinant protein(s) may be produced intracellularly or secreted into the culture medium from which it can be recovered and/or collected.

After inoculation of the medium with a eukaryotic cell engineered to recombinantly express an exogenous protein, the cell is cultured under fermentation conditions.

Any fermentation cell culture method or system that is amenable to the growth of the cells for biomass generation and expression of recombinant protein may be used. For example, the cells may be grown in batch or fed-batch cultures, where the culture is terminated after sufficient expression of the recombinant protein has occurred, after which the protein is harvested and, if required, purified. Fluidized bed bioreactors, hollow fibre bioreactors, roller bottles, shake flasks or stirred tank bioreactors may be used and operated in a batch, fed-batch, continuous, semi-continuous or perfusion mode. Fed batch culture is a widely-practiced culture method for large scale production of proteins from eukaryotic cells. See e.g. Chu and Robinson (2001), Current Opin. Biotechnol. 12: 180-87. If a fed-batch culture is used, feeding of the culture may take place continuously, or periodically during culture. When multiple feeds are given, these may be given daily, every other day, every two days etc., more than once per day, or less than once per day, and so on, with the same or different feeding solutions for each feed.

In one aspect of the method for producing a recombinant protein of the present invention, the method further comprises the step of recovering the recombinant protein from the cell culture.

In one embodiment the expressed recombinant protein is recovered from the cell culture supernatant. In one embodiment, the recombinant protein is recovered from the cells. Recovery of the expressed protein either during or at the end of a culture period, typically the production phase, can be achieved using methods known in the art. The expressed protein may be isolated and/or purified as necessary using techniques known in the art, such as protein A columns, ion exchange column purification and/or size exclusion column purification.

### Examples

The following descriptions underlie the information provided in the examples.

Viable Cell Densities, Viability and Cell Time Integral

For analysis of viable and total cell densities an automated Cedex HiRes system (Roche Diagnostics, Mannheim, Germany) was used. Discrimination between viable and total cell densities were evaluated using the trypan blue exclusion staining method and analyzing more than 10 pictures per sample and day according to the manufacturer's specifications.

Viable cell density (VCD) and cell viability were calculated as described in equation 2 (Equ 2) and equation 3 (Equ 3), respectively.

Viable cell density=N_{Trypan blue negative}×(10⁵ viable cells/ml) (Equ 2)

Cell viability=N_{Trypan blue negative}/(N_{Trypan blue negative}+N_{Trypan blue positive} cells)×100% (Equ 3)

As indicator for overall biomass generation in the process a cumulative cell time integral (CTI) was calculated using equation 4 (Equ 4):

Cell time integral=□(0.5×(VCDₙ₋₁+VCDₙ)×(tₙ−tₙ₋₁))×(10⁵ viable cells×d/ml) (Equ 4)

In the above equations, N=Cell Number, tₙ=time point n; and tₙ₋₁=time point one before n; VDCₙ=Viable cell density at time point n; VDCₙ=Viable cell density at one time point before n; CTI=Cell time integral; dₜₙ=Diameter cells at time point n; ml=milliliter and d=day.

Capillary Electrophoresis (CE-SDS)

CE on a chip was performed with the Agilent Bioanalyzer 2100 or the PerkinElmer LabChip GX and the respective protein kits according to the instructions of the manufacturer.

Quantification of Product Titer and Calculation of Cell Specific Productivity (gP)

Product titer war quantified by a Cobas Integra 400 plus system (Roche, Mannheim, Germany) according to the manufacturer's protocol. The overall cell specific productivity (gP) was calculated for the analysis of cell production capacity according to equation 5.

qP=(Titerₙ−Titerₙ₋₁/(CTIₙ−CTIₙ₋₁)×(pg/(viable cell×d)) (Equ 5)

Wherein CTI is cell time integral, n is as defined above, ml=milliliter and d=day

HCP Assay

The Chinese hamster ovary host cell protein (CHO HCP) content in process samples is determined by an electrochemiluminescence immunoassay (ECLIA) on a Cobas e 411 immunoassay analyzer (Roche Diagnostics). The assay is based on a sandwich principle using polyclonal anti-CHO HCP antibody from sheep.

First incubation: CHO HCP from 15 μL sample (neat and/or diluted) and a biotin-conjugated polyclonal CHO HCP specific antibody form a sandwich complex, which becomes bound to streptavidin-coated microparticles via interaction of biotin with streptavidin.

Second incubation: After addition of polyclonal CHO HCP-specific antibody labeled with ruthenium complex (Tris(2,2′-bipyridyl)ruthenium(II)-complex) a ternary sandwich complex is formed on the microparticles.

The reaction mixture is aspirated into the measuring cell where the microparticles are magnetically captured onto the surface of the electrode. Unbound substances are then removed in a washing step. Application of a voltage to the electrode then induces chemiluminescent emission which is measured by a photomultiplier.

The concentration of CHO HCP in the test sample is finally calculated from a CHO HCP standard curve of known concentration.

PLBL2 Assay

The CHO Phospholipase B-like 2 protein (PLBL2) content in process samples is determined by an ECLIA on a Cobas e 411 immunoassay analyzer (Roche Diagnostics). The assay is based on a sandwich principle using monoclonal anti-CHO PLBL2 antibody from mouse.

In a first incubation step, CHO PLBL2 from 30 μL sample (neat and/or diluted), biotin labeled monoclonal CHO PLBL2-specific antibody, and a monoclonal CHO PLBL2-specific antibody labeled with a ruthenium complex (Tris(2,2′-bipyridyl)ruthenium(II)-complex) form a sandwich complex.

In a second step after addition of streptavidin-coated microparticles, the ternary complex becomes bound to the solid phase via interaction of biotin and streptavidin.

The reaction mixture is aspirated into the measuring cell where the microparticles are magnetically captured onto the surface of the electrode. Unbound substances are then removed in a washing step. Application of a voltage to the electrode then induces chemiluminescence, which is measured by a photomultiplier.

The concentration of CHO PLBL2 in the test sample is finally calculated from a CHO PLBL2 standard curve of known concentration.

Clusterin Assay

The residual Clusterin content in process samples is determined by a commercial assay from Merck Millipore (GyroMark HT Kit GYRCLU-37K) which was used according to the manufacturer's instructions. In brief, this assay is a Sandwich ELISA based, sequentially, on:
1) binding of the rat Clusterin biotinylated capture antibody to the streptavidin coated affinity columns of the Bioaffy 1000 nL CD,
2) capture of rat Clusterin molecules from samples to the anti Clusterin antibody,
3) binding of a second dye-labeled anti Clusterin detection antibody to the captured molecules, and
4) quantification of the rat Clusterin using the Gyrolab Evaluator.

Trace Element Solutions

Redox active trace metals are added to the base medium singly or in the form of a Trace Element Solution (TES), e.g. having the following compositions:
TES1: iron, copper, chromium, cobalt, selenium, manganese, vanadium and molybdenum.
TES2: iron, copper, selenium, manganese, vanadium and molybdenum.
TES3: copper, selenium, manganese, vanadium and molybdenum.

Any redox active trace metals not included in a TES may be added separately to the medium. For example, if TES3 is used, iron may be added separately.

The C_{TES} for TES1, TES2 and TES3 is calculated using equation 1, above.

Inoculation Conditions

In the Examples the inoculation conditions were: a pH of about pH 6.0 to about pH 8.0, a temperature of about 20° C. to about 39° C. and a % O₂ of about 10% to about 80%.

DoE Experiments

Statistical experiment planning by Design of Experiments (DoE) is a powerful and well-known technique especially for biological and chemical reaction optimization intentions where many factors interdependently influence the result. For example, DoE was successfully used for optimization of cell culture media (Zhang et al. 363-78), fermentation processes (Fu et al. 1095-105), protein purification processes (Pezzini et al. 8197-208) and, cell culture conditions (Chen et al. 1211-21).

Based on the desired outcome different DoE approaches can be applied to the present process. For example, the Full factorial design is well suited for situations, where 1) only few factors are considered, 2) we are interested mainly in linear effects and 3) a large number of experiments is feasible. Since here we were interested in quadratic effects, we used a face-centered central composite design. Central composite designs extends the classical design points by axial points to enable the estimation of curvature of quadratic effects.

All DoE approaches in this study were planned and analyzed using statistical software tool JMP (SAS Institute GmbH, Böblingen, Germany). Following the response surface methodology (RSM) we have fitted the response for cell-specific productivity qP to a second-degree polynomial model and obtained significant results in all cases. The simulation uses the ‘Prediction Profiler’ feature of the JMP software.

### Example 1

Screening and Assessment of Effect of Treatment of Medium During Conditioning on Cell-Specific Productivity and Host Cell Protein Content—Interaction Effect Screen for Addition of Redox-Active Trace Metals, Cystine and Media Supplementation Timing

In a first experiment, we aimed to screen the relevance and effect of different concentrations of redox-active trace metals, cystine and the timing of media supplementation on cell culture performance and product quality to test our hypothesis that cell culture performance can be improved if the medium supplemented with redox-active trace metals and cystine, cysteine or a cysteine-derivative is held under appropriate conditions for a period of time before inoculation with cells producing recombinant protein. For that, we used a state-of-the-art statistical design-of-experiment (DoE) approach using a respective ambr15 robotic cell cultivation screening system. We applied a central composite screening design DoE using trace metals, cystine and supplementation timing. Redox active trace metals were added to the medium using a TES in a cumulative amount of C_{TES}=6.39 μM (referred to as “low TES”) to 320 μM (referred to as “high TES”). Cystine was added to the medium, reaching a final concentration in the medium of about 0.5 mM (referred to as “low CysCys”) to 8 mM (referred to as “high CysCys”).

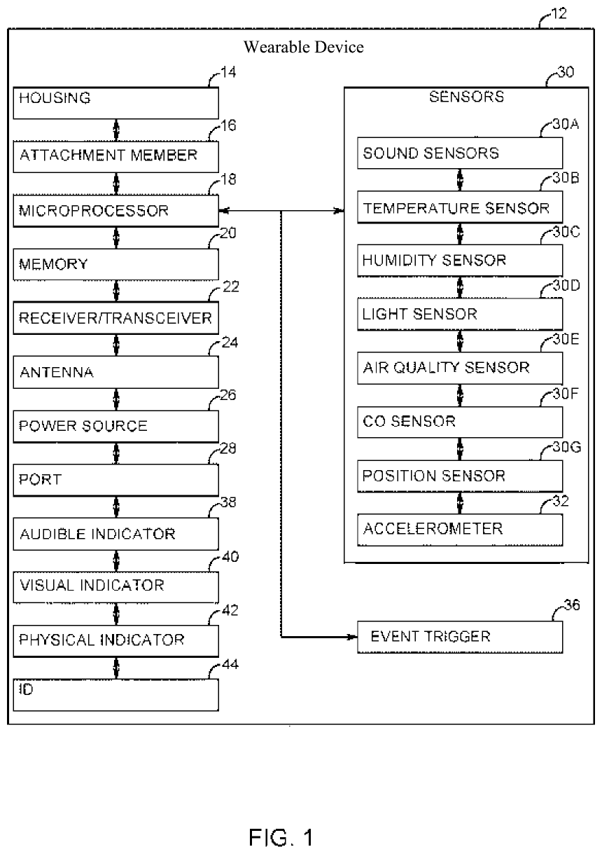

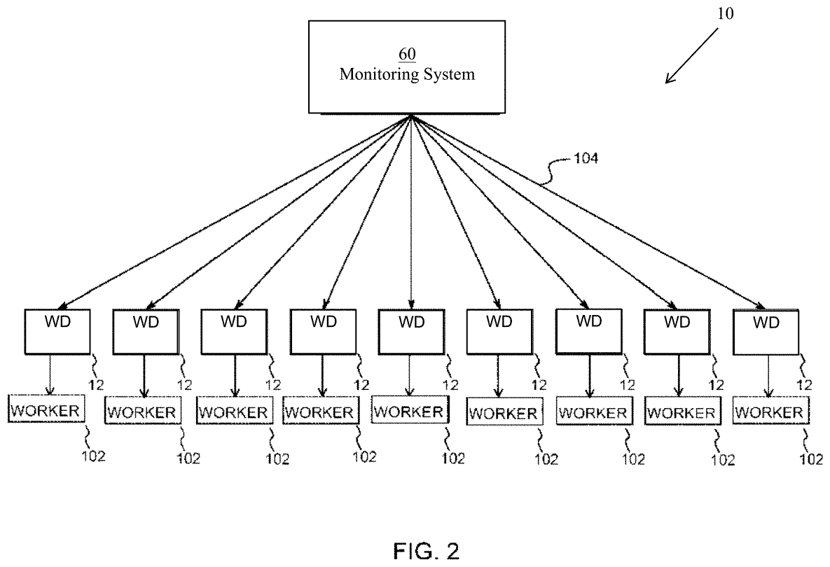

The variation of all parameters between a “high” and a “low” level show the relevance of input variables and respective results for cell-specific productivity (qP) (FIG. 1) and CHO cell derived host cell protein impurities (CHO HCP) (FIG. 2). FIG. 1 shows that qP typically increases with an increase in the length of medium conditioning before inoculation with cells. FIG. 2 shows that the amount of host cell protein impurities decreases when the conditioning period before inoculation is extended. Thus, the statistical experimental setup shows that cell culture performance benefits from conditioning the supplemented medium before inoculation and from increasing the length of the conditioning period. More detailed analyses are described in Examples 2 and 3.

### Example 2

Assessment of the Effect of the Addition of Nutrients to the Medium Before the Start of Conditioning on Cell-Specific Productivity, HCP Impurity Load and Process-Specific Oxygen Demand

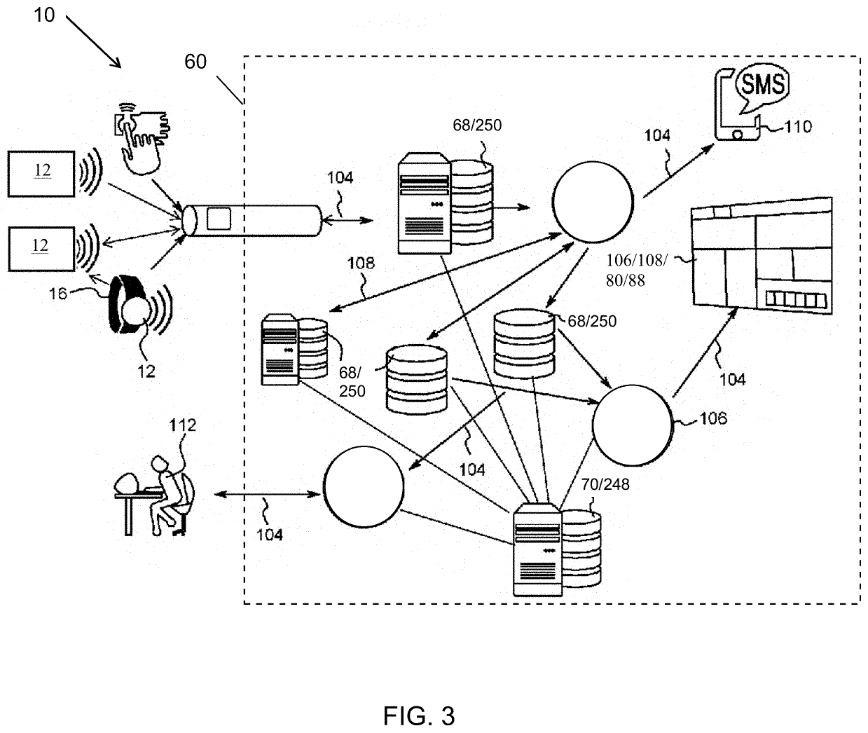

In this example, the method comprised culturing different CHO-K1 cell lines (clones) in a 14 day fed batch culture using fully controlled 2 L bioreactor systems under standard conditions. In this example, a standard cell culture medium (chemically defined base medium) was loaded to a 2 L bioreactor held at inoculation conditions and then supplemented with trace metals and cystine. The conditioning period then started. The base medium contained no cysteine or its derivatives and further did not include any of the trace metals used for supplementation. Redox active trace metals were added to the medium in a cumulative amount of C_{TES}=53.9 μM. Cystine was added to the medium, reaching a final concentration in the medium of about 3 mM. After attainment of inoculation conditions, the supplemented medium was held in the bioreactor under inoculating conditions for 24 hours prior to inoculation (see FIG. 3, “Aged”). As a comparison, the same base medium was supplemented with the same amounts of the same trace element solution and cysteine and was also inoculated at the same time with the respective clones before being held under the same inoculation conditions (see FIG. 3, “Fresh”).

For simplicity, the term “Aged” when used in the examples refers to the results determined from the experiments in which the medium was treated according to the invention; whereas the term “Fresh” when used herein refers to the results determined from the experiments in which the medium was the same in all respects as the “Aged” medium, but was inoculated directly after supplementation with the nutrients.

Both, the “Aged” and the “Fresh” cultures of the same clones were run under the same conditions.

In a first set of experiments, three CHO clones expressing different monoclonal antibodies were used. Clones 1 and 2 express different bispecific IgG1 antibodies and clone 3 expressed a bispecific antibody Fab fragment. The respective host cell lines (HCL) were also used.

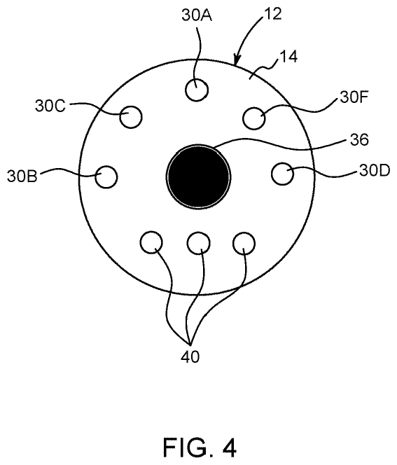

Cell culture and product characteristics were analysed by the methods described above. Results are shown in FIGS. 4A to 12B.

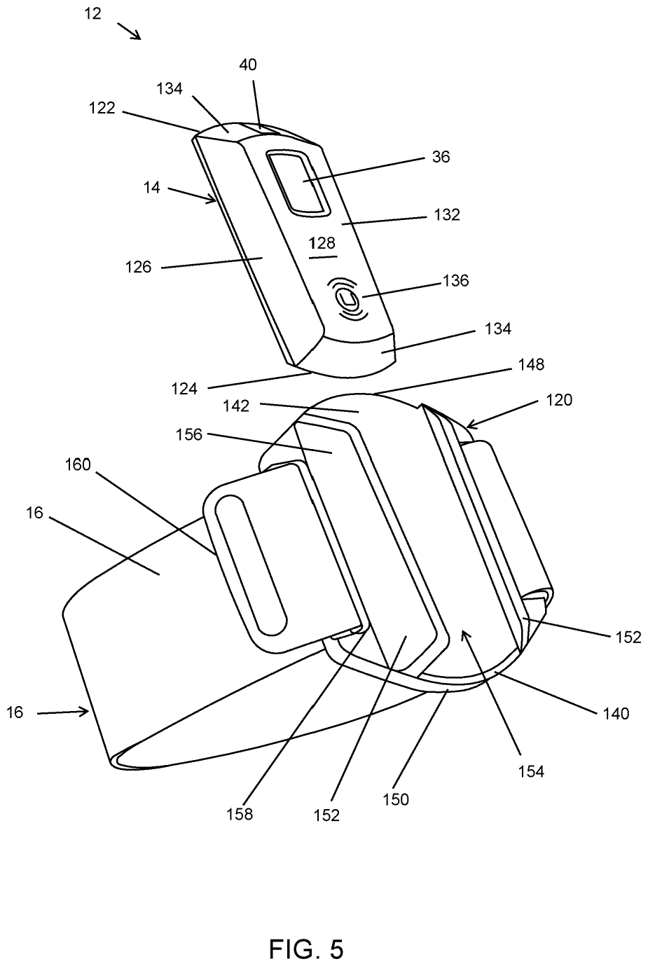

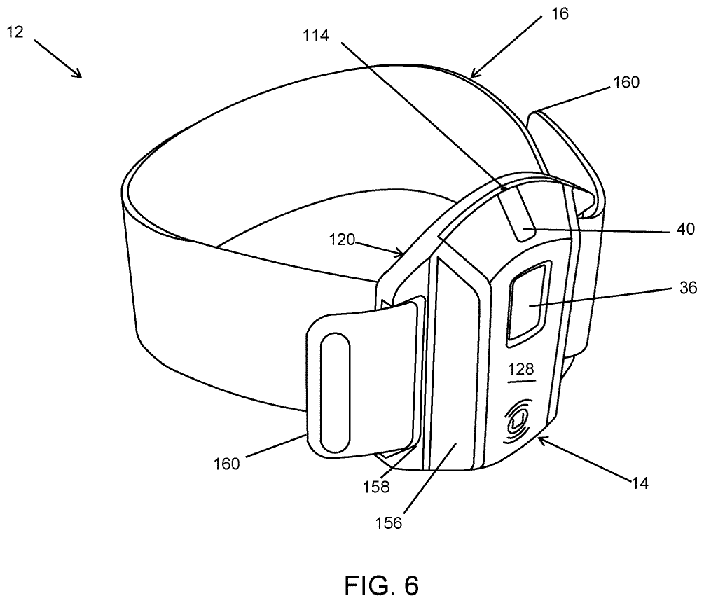

Medium treatment according to the invention resulted in a reduced cell number for all clones and the respective HCL at the end of fermentation (FIG. 4A to 4D). Medium treatment did not impact product titer (FIG. 5A to 5C).

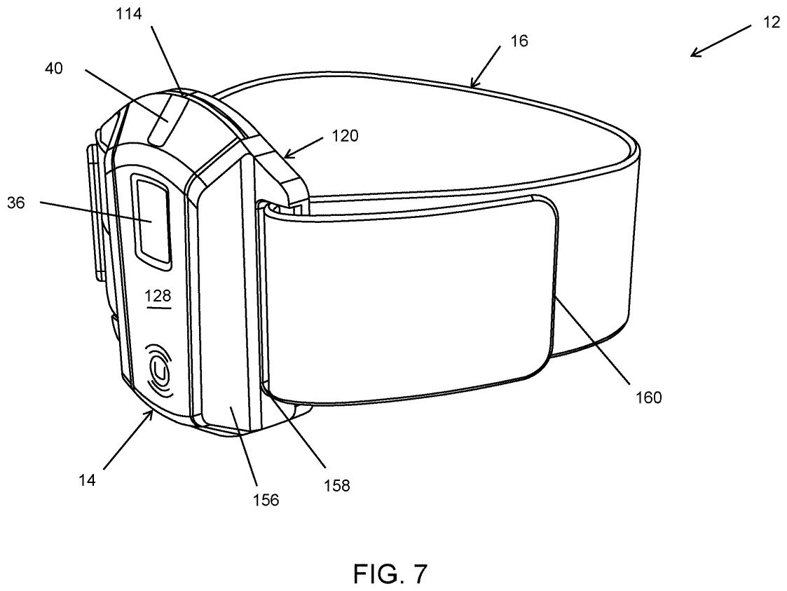

Medium treatment according to the invention results in an 18-27% higher cell-specific productivity (FIG. 6A, C, E and FIG. 7A, B). By plotting the cell-volume-integral vs product titre, comparable biomass, and therefore cell volume-specific productivity was observed for all three tested clones (FIG. 6B, D, F).

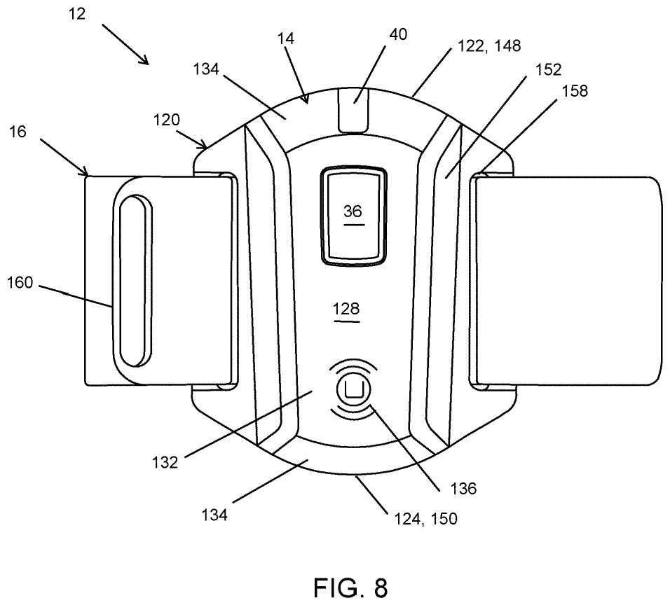

In addition, cell culture in medium treated according to the invention exhibited a decreased global demand for oxygen in the process, yet the biomass is the same as for the cells cultured in medium supplemented with the same nutrients and directly inoculated (FIG. 8A to 8F).

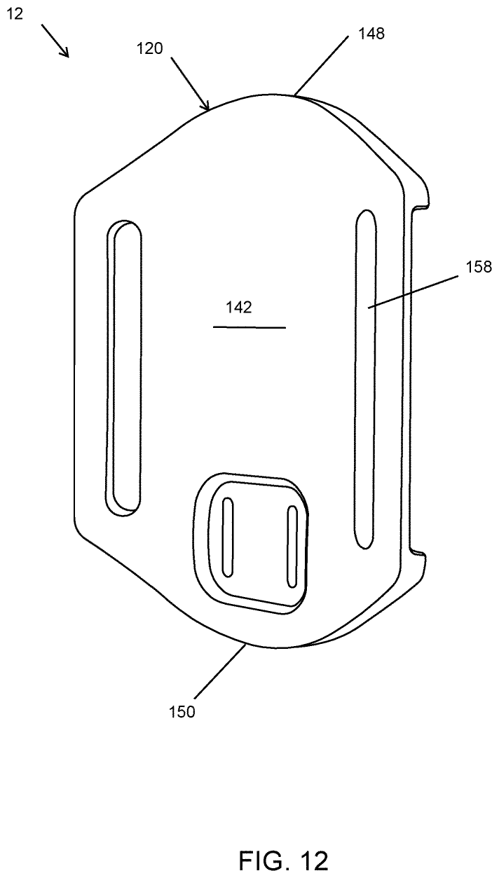

Medium treatment did not impact product quality at day 14, as measured by CE-SDS (capillary electrophoresis sodium dodecyl sulphate) and size exclusion chromatography (SEC) (FIGS. 12A and 12B).

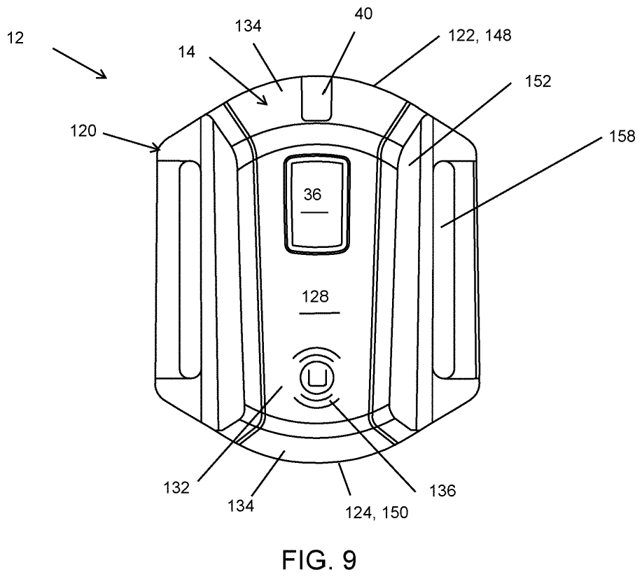

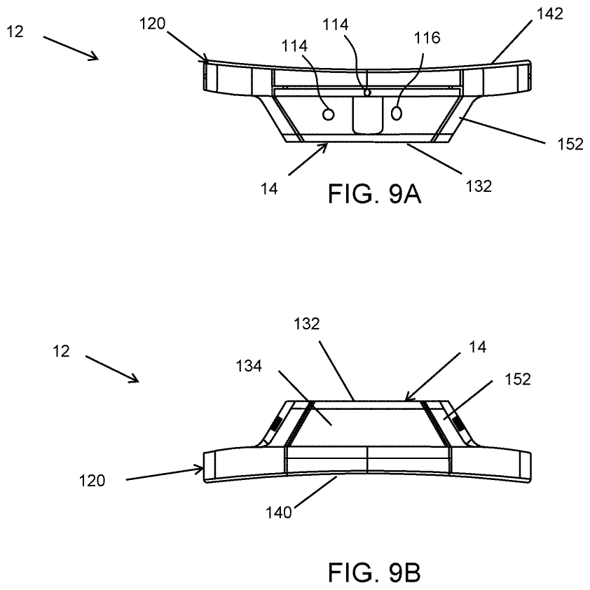

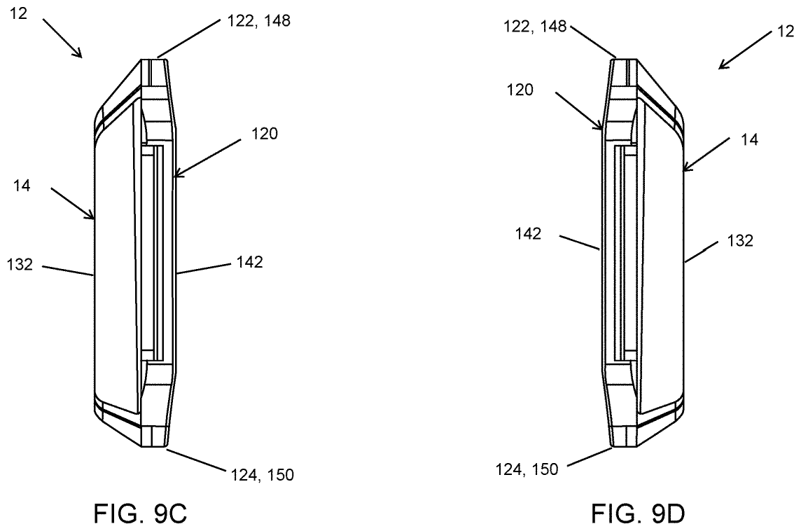

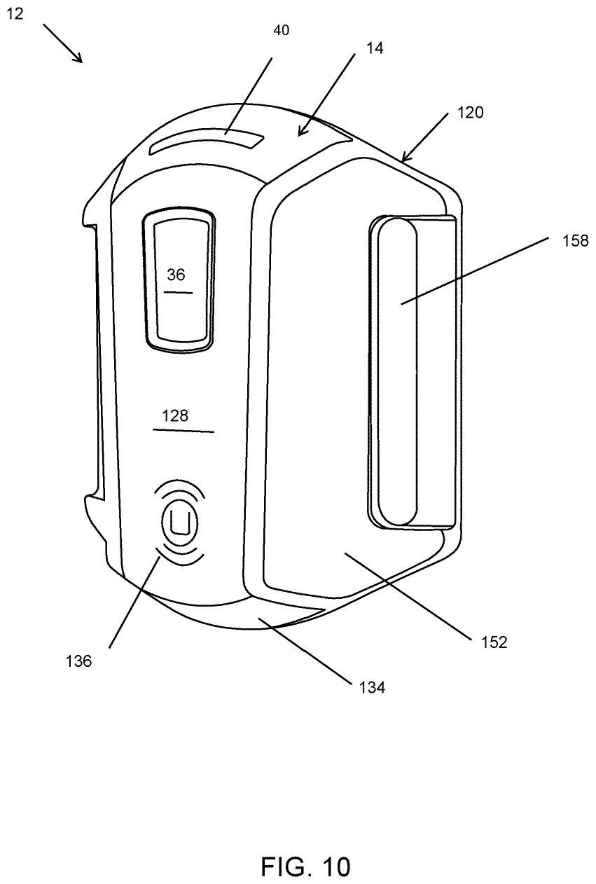

Furthermore, supernatant of the cultures at day 14 were used to analyse the amount of host cell protein (HCP) load, which is critical for subsequent down-stream processing (DSP) efficiency. By that, a tendency for reduced LDH activity in the medium (up to 26%), a surrogate for dead cells, and global HCP level (up to 54%) were observed in a medium treated according to the invention (FIGS. 9A, 10A and 9B, 10B). For a more specific HCP, PLBL2, the same tendency was observed and

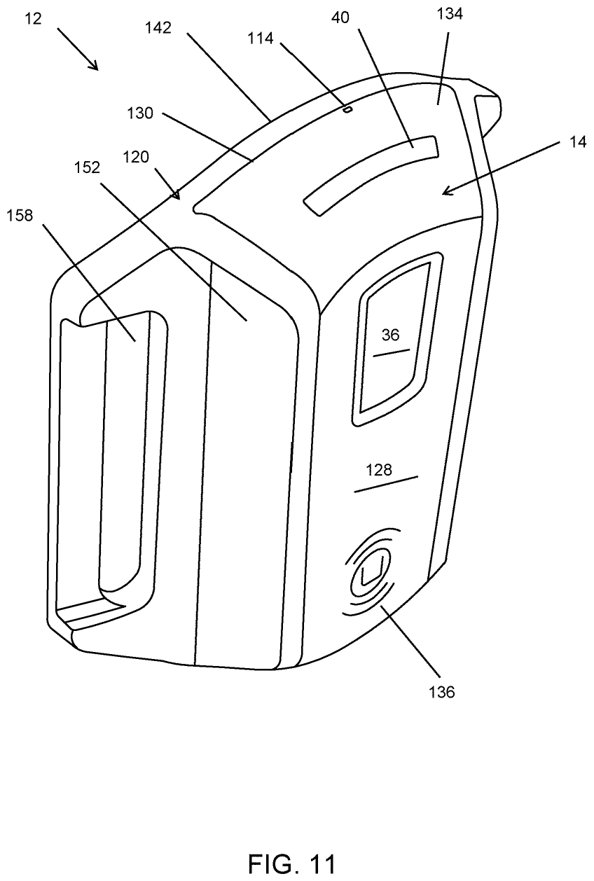

PLBL2 load was reduced between 17-40% by using medium treated according to the invention (FIGS. 11A and 11B).

### Example 3

Assessment of the Effect of the Duration of the Conditioning Period on Cell-Specific Productivity and Process-Specific Oxygen Demand

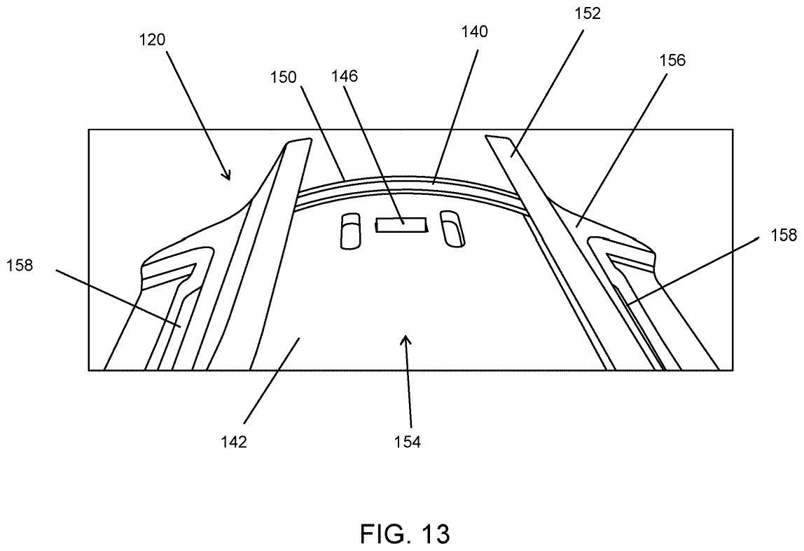

The base medium of Example 2 was used in this Example, with supplementation thereof with trace metals and cystine at different times, as depicted in Scenario 2, FIG. 13. In all experiments, the base medium was held at inoculation conditions for 72 hours. The timing of supplementation differed in each setting. In one setting, the base medium was supplemented with trace metals and cystine before the start of conditioning. Subsequently the supplemented medium was held under inoculating conditions for 72 hours prior to inoculation (see FIG. 11, “−72 h”). In another setting, the base medium was supplemented with trace metals and cystine approximately 24 hours after the start of conditioning. Subsequently the supplemented medium was held under inoculating conditions for 48 hours prior to inoculation (see FIG. 11, “−48”). In yet another setting, the base medium was supplemented with trace metals and cystine approximately 48 hours after the start of conditioning and the supplemented medium was then held under inoculating conditions for 24 hours prior to inoculation (see FIG. 11, “−24 h”). In each setting, redox active trace metals were added to the medium in the form of a TES in a cumulative amount of C_{TES}=53.9 μM; and cystine was supplemented to the medium to reach a final concentration of 3 mM.

In this second set of experiments, inoculation was with cells of clone 2, as defined above. The experiment was performed in a 14 day fed-batch cultivation using fully controlled 2 L bioreactor systems. The case “−24 h” was run in triplicate to analyze the variance of the experiment. The relative standard deviation was leveraged to the other cases.

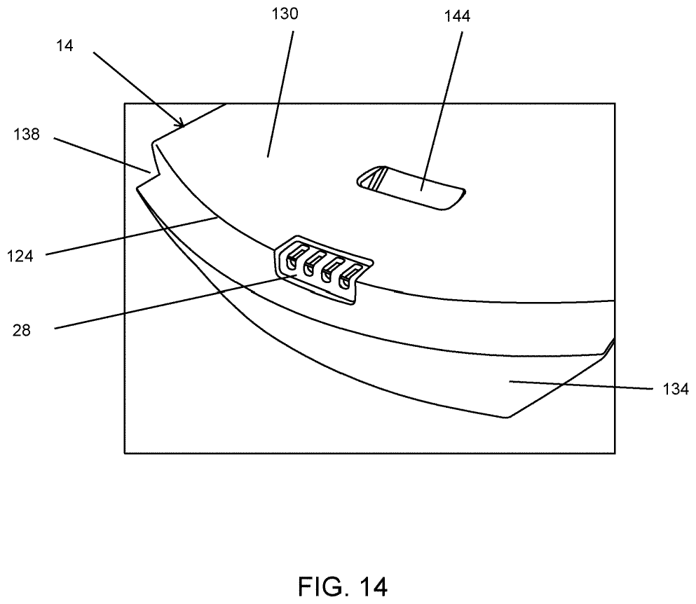

Cell culture and product characteristics were analysed by the methods described above. Results are shown in FIGS. 14 to 17.

Product titer during the cultivation is shown in FIG. 14.

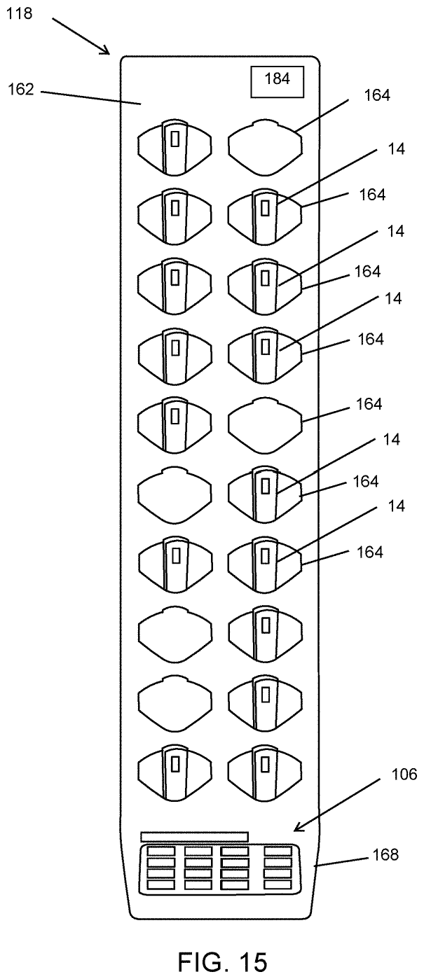

Cell specific productivity increased the longer the supplemented medium was held under inoculation conditions (FIG. 15).

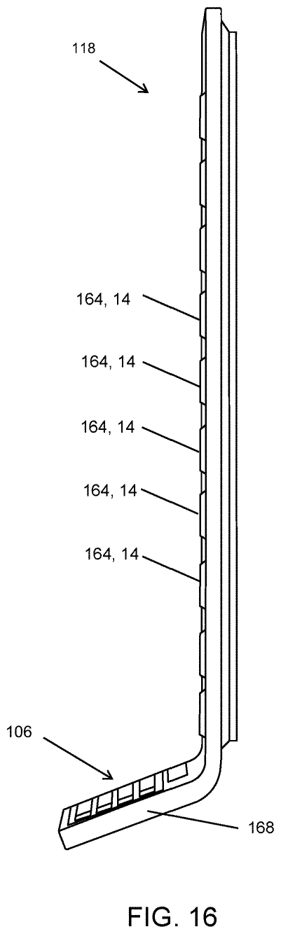

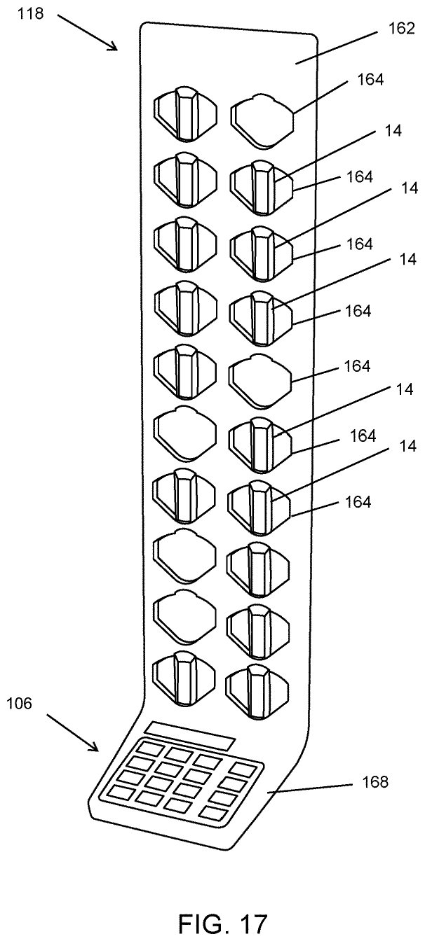

Oxygen demand is indicated in FIGS. 16 and 17.

### Example 4

Time-Specific Use of Redox-Active Trace Metal and Cystine Media Supplementation Timing for Tailored Cell Growth and Cell-Specific Productivity Regulation in N-1 and N Phases

The effect of the timing of redox-active trace metal and cystine media supplementation was analyzed using shake flask inoculation cultures and 14 day fed-batch production cultures with 9 fully controlled bioreactors of an ambr250™ robotic bioreactor system. Medium for the N-1 phase and/or for the production phase were supplemented and held under inoculation conditions for different time periods. “Aged” medium was supplemented and conditioned for between 12, 24, 48 and 96 hours pre-inoculation and “Fresh” medium was supplemented directly (0 hours) before inoculation. The detailed planning of the experiment is shown in Table 1. Possible further medium-tailoring scenarios are described in FIG. 18.

TABLE 1
Timing of trace metal and cystine supplementation
and conditioning before inoculation in hours for
N-1 (“growth media”) and for N (“production media”).

| | Growth Media (for N-1) | Production Media (for N) |
|---|---|---|
| | Supplementation time | Supplementation time |
| | before inoculation in | before inoculation in |
| Bioreactor | hours | hours |
| #1 | 0 | 0 |
| #2 | 12 | 0 |
| #3 | 24 | 0 |
| #4 | 48 | 0 |
| #5 | 12 | 12 |
| #6 | 12 | 24 |
| #7 | 24 | 24 |
| #8 | 0 | 48 |
| #9 | 0 | 96 |

Clone 2 from Example 2 was used for the study. The cells were thawed and grown in seed train in a standard cell culture medium (proprietary chemically defined medium; CDM1) for two weeks in shake flasks to ensure stabilization of cell growth and to ensure consistent cell doubling time. One week before inoculation of the production (N) phase cells were transferred to another standard cell culture medium (proprietary chemically defined medium; CDM2) to ensure expansion of cells (stage N-2). Four days before inoculation of the N-phase cells were transferred into temporally different, final supplemented CDM2 media obtained by adding redox-active trace metals and cystine at 0 h (Fresh), or 12 h, 24 h and 48 h (Aged) before inoculation (see Table 1) for the N-1 phase. These N-1 “pre-cultures” were then used for inoculation of a 14-day production (N) fed-batch process. CDM2 media was used for the N-phase with temporally different final supplementation of cystine and redox-active trace metals at 0 h (Fresh), or 12 h, 24 h 48 h or 96 h (Aged) before inoculation (Table 1). To avoid limitation of substrates during the N-phase, the cells were fed by a continuous proprietary feed 1 (start at day 3 until harvest) and adjustment of glucose to target values.

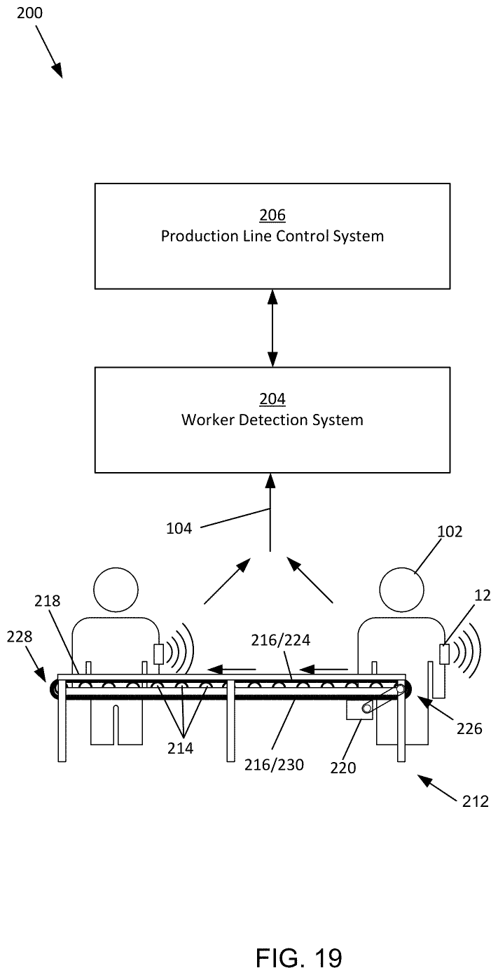

When the “Aged” supplemented medium was used in the growth phase, the result was a reduced cell number, illustrated by approximately 10% less viable cells (FIG. 19) as compared to the use of a “Fresh” medium for the growth phase. These results indicate that cell growth and production of biomass is better when the growth phase is performed in a “Fresh” medium and that “Ageing” of the medium for the growth phase for any time period does not benefit cell growth.

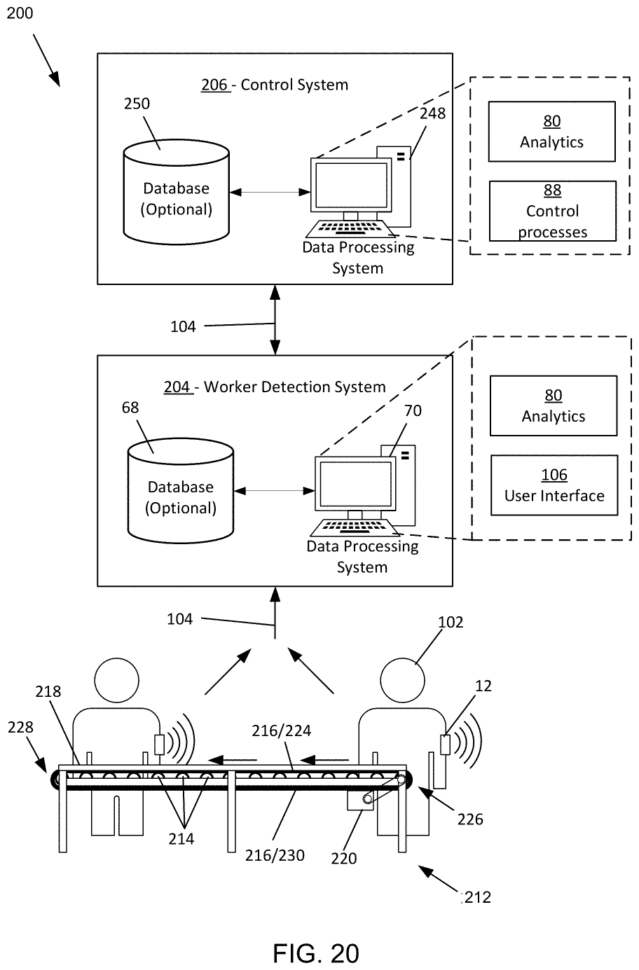

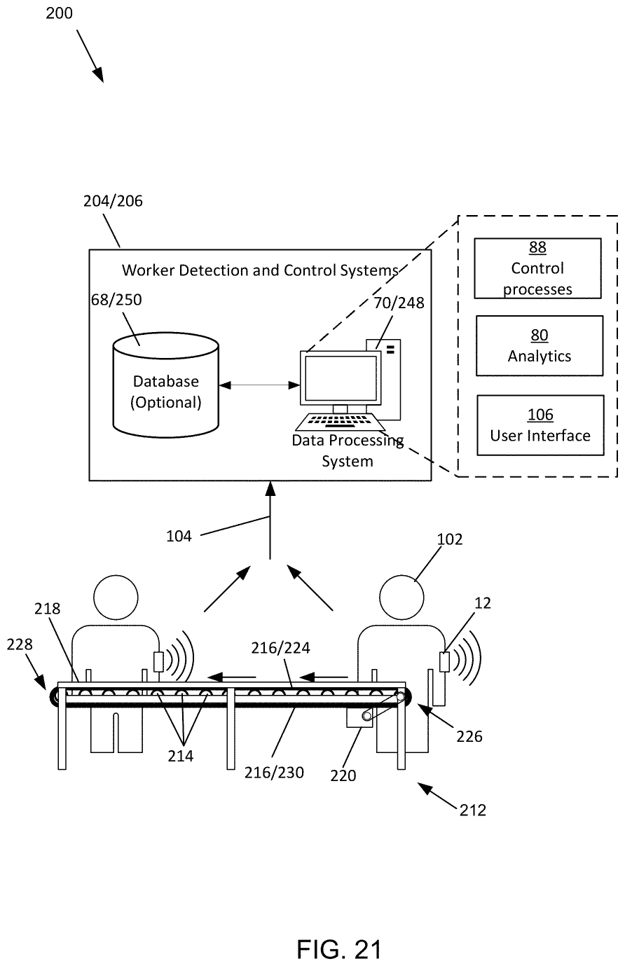

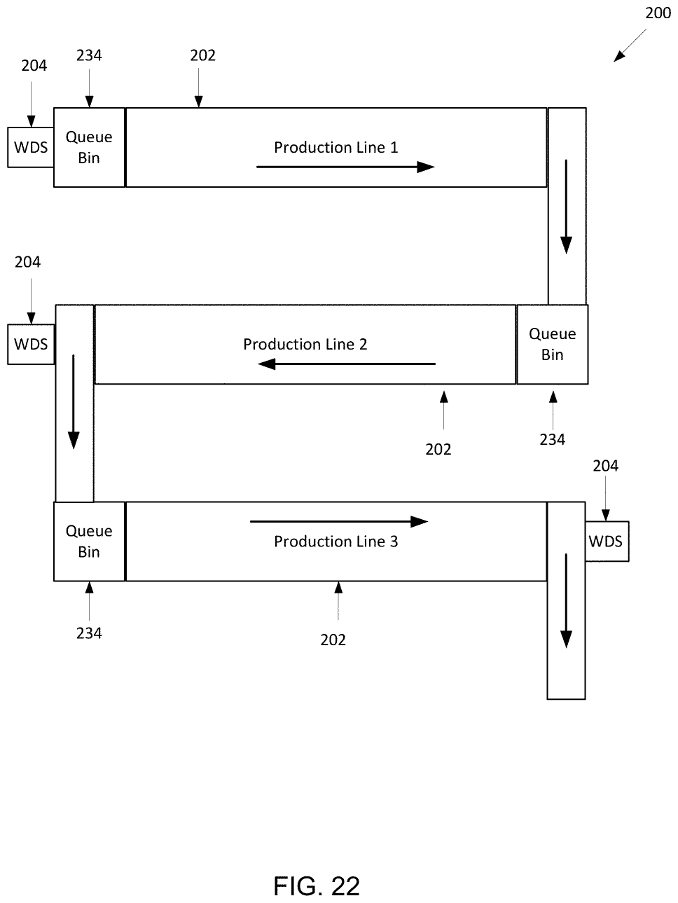

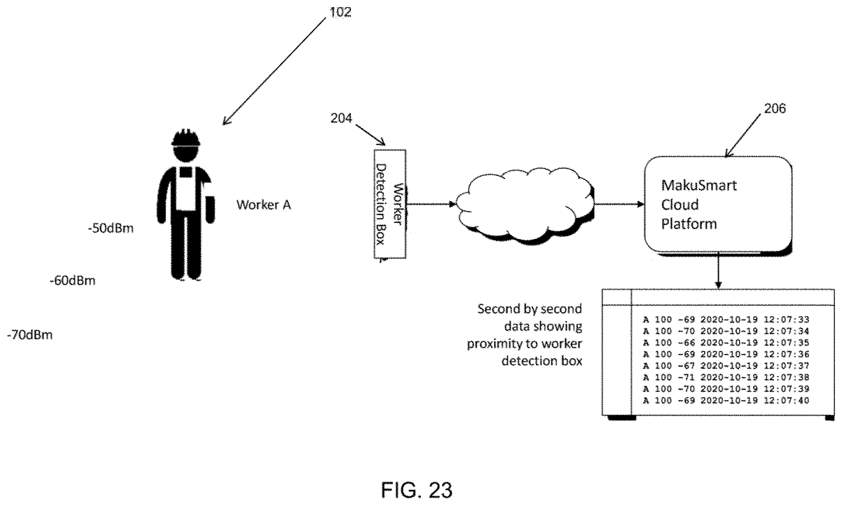

In the subsequent 14-day production phase, which was performed as a fed-batch fermentation, all cases show comparable titer (FIG. 21), and product quality as analyzed by CE-SDS (FIG. 22) and SEC (FIG. 23). In contrast, the cell-specific productivity of the cells was increased either by using cells from “Aged” media for the N-1 phase and “Fresh” media for the N phase (black bars in FIG. 20) or using the “Aged” media for the N phase and “Fresh” media for the N-1 phase (dotted bars in FIG. 20). Interestingly and unexpectedly, the effect of increased cell-specific productivity (measured only in N stage) resulting from culture in the “Aged” medium was inherited from those N-1 cultures by the cells in the N-phase carried out in “Fresh” supplemented media. It was noted that using “Aged” media in both the N-1 and N phases seems to have a negative effect on cell-specific productivity, with an increase in the conditioning time after supplementation for media in both the N-1 and N phases causing a progressive decrease in specific productivity (striped bars in FIG. 20).

The supernatant from the N-phase was also used to analyze the impact of media “ageing” on host cell protein impurities. For that, the “content” (i.e. concentration of host cell impurity per concentration target protein) of global host cell protein, clusterin and PLBL2 (phospholipase B-like 2) was analyzed as described in equations 6 to 8.

$$Host Cell Protein Content = \frac{{c}_{Host Cell Protein}}{{c}_{Target Protein Titer}} * \frac{ng}{mg} \qquad ( Equation 6 )$$

$$Clusterin Content = \frac{{c}_{Clusterin}}{{c}_{Target Protein Titer}} * \frac{ng}{mg} \qquad ( Equation 7 )$$

$$PLBL 2 Content = \frac{{c}_{Host Cell Protein}}{{c}_{Target Protein Titer}} * \frac{ng}{mg} \qquad ( Equation 8 )$$

$$c : concentration$$

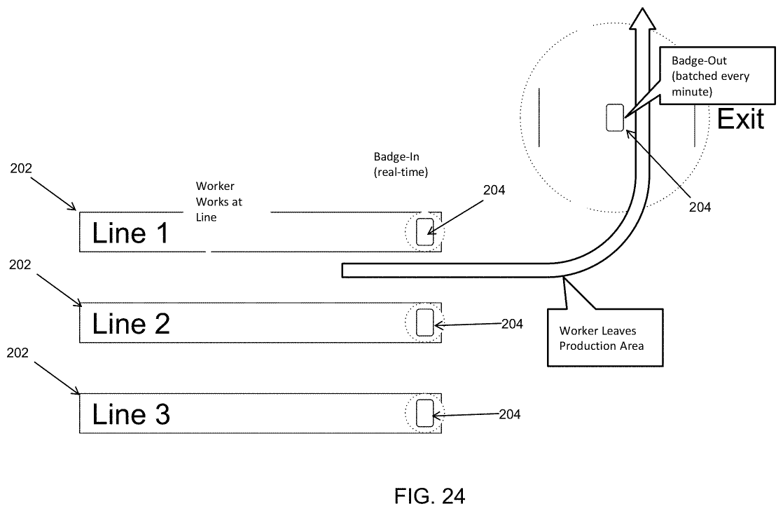

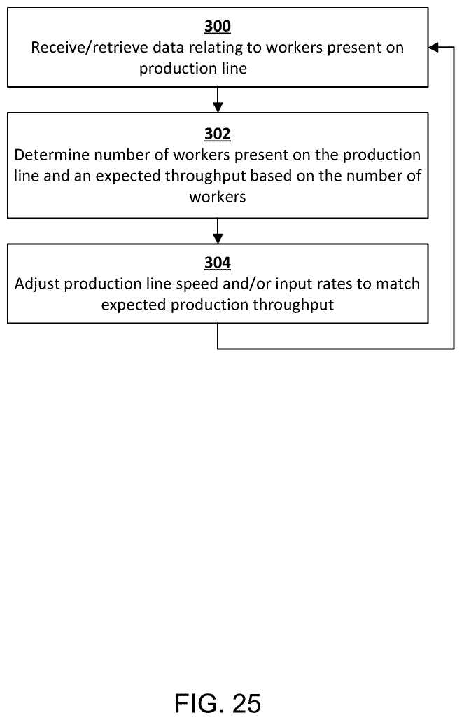

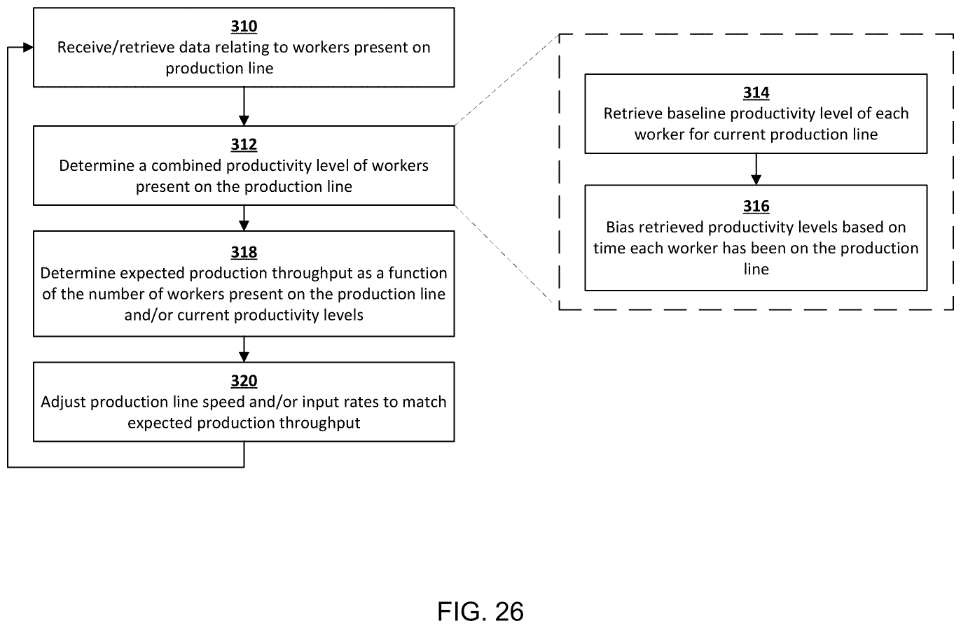

Ageing of media for use in both N-1 and N phases causes a slight reduction in HCP content (FIG. 24), and Clusterin load (FIG. 25). No effects on PLBL2 were detected in this experiment (FIG. 26).

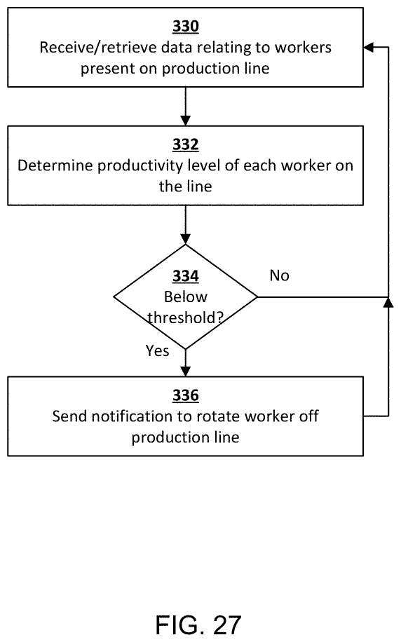

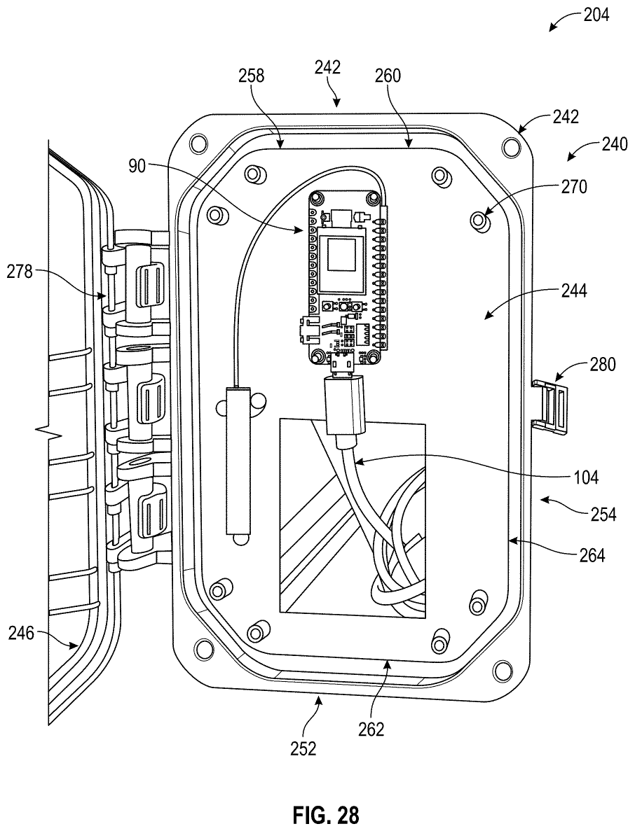

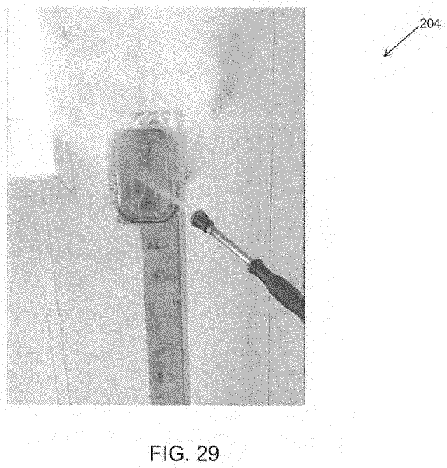

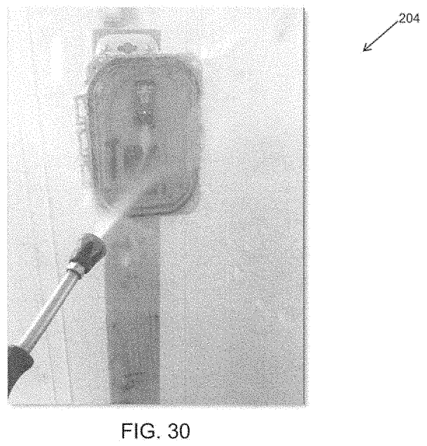

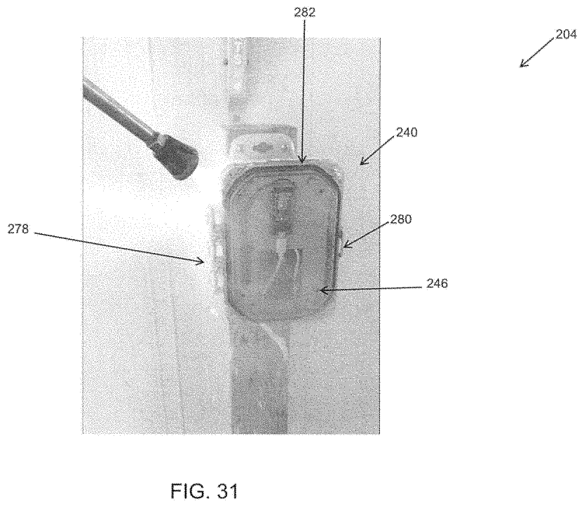

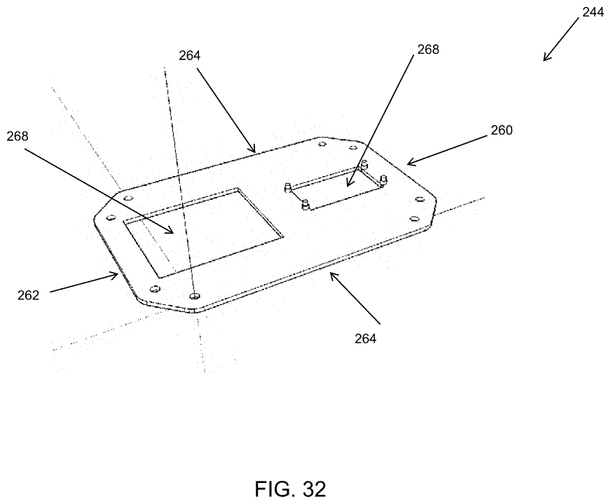

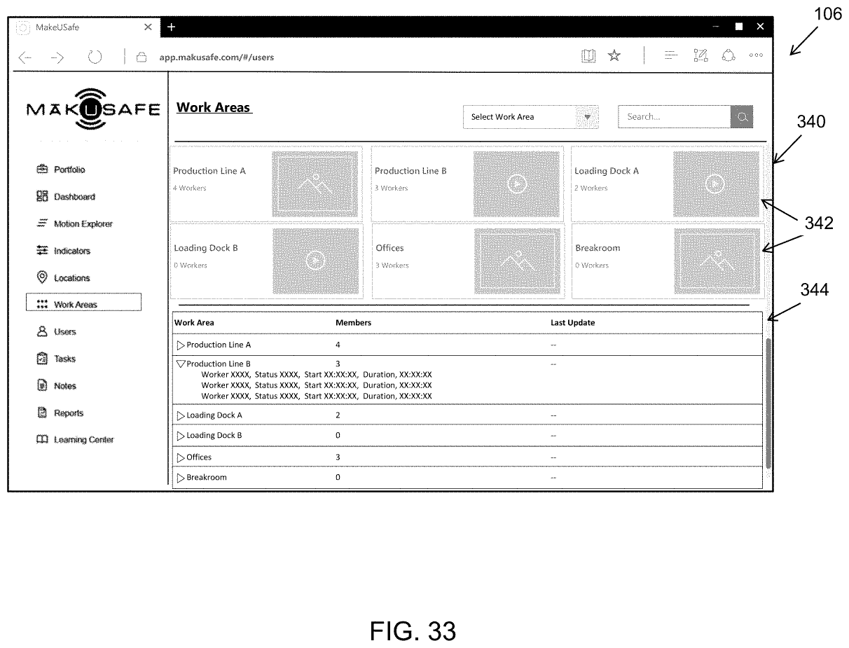

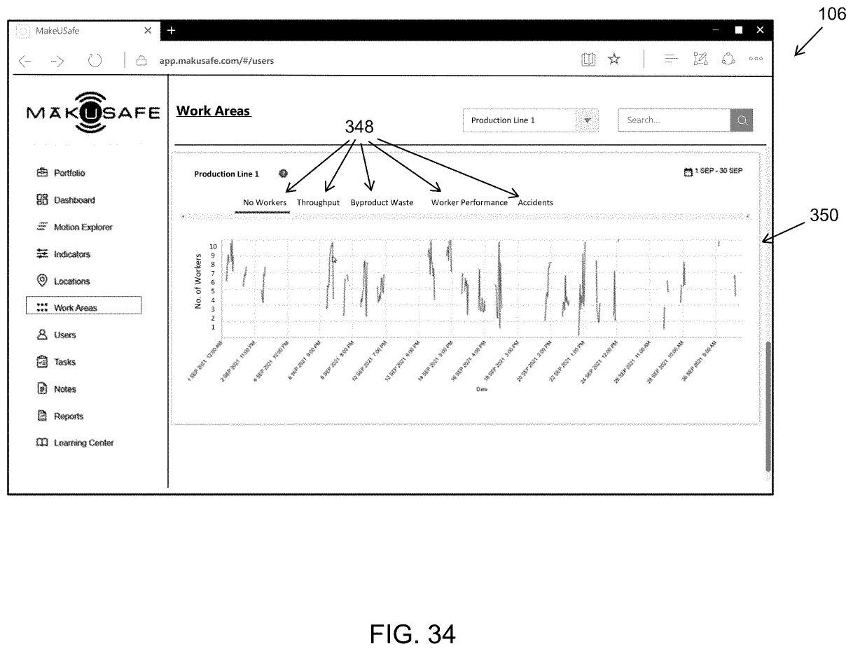

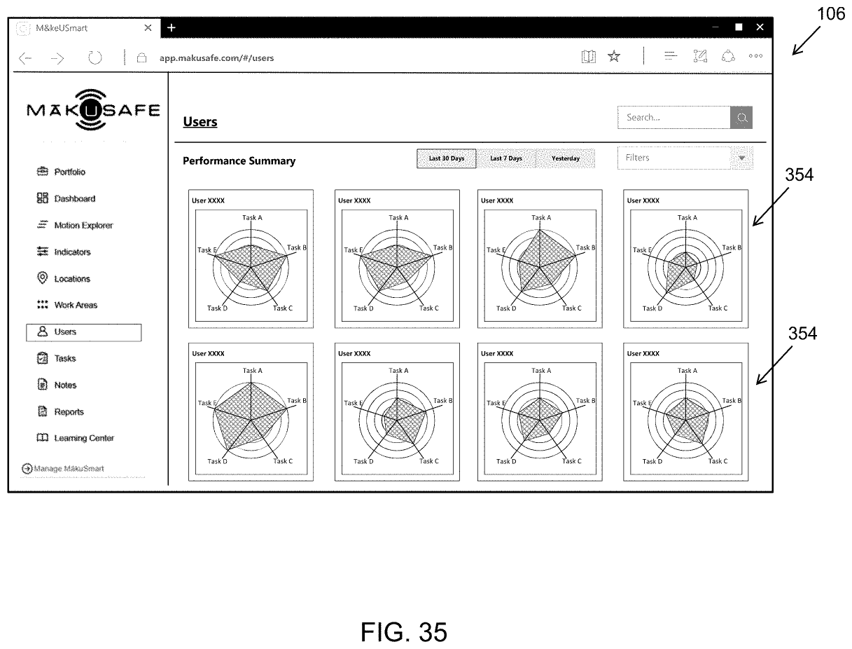

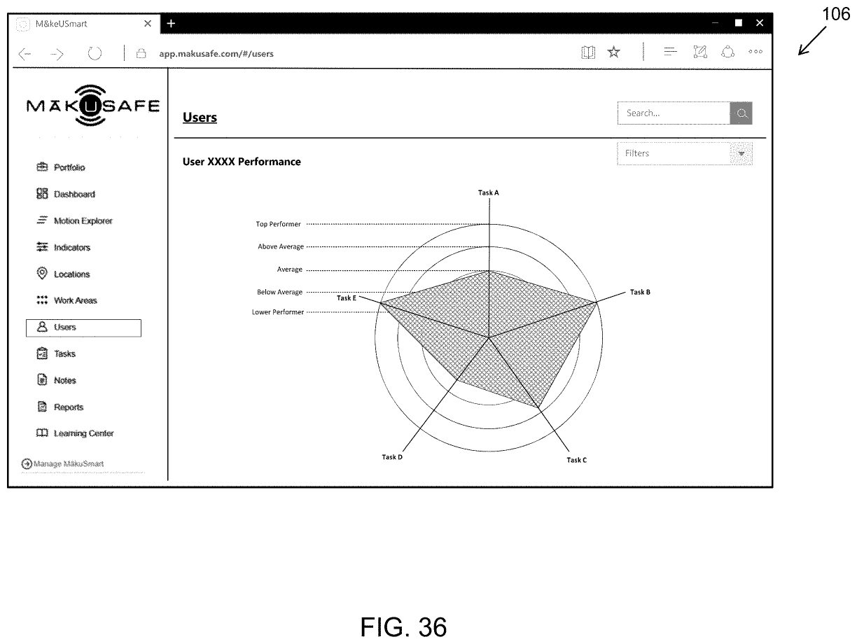

Using “Aged” media and increasing the conditioning period after supplementation decreased the oxygen demand of the 14-day fed-batch culture yet, due to technical limits the effect was not as strong as in larger bioreactors with higher gas flows (lower gas flows in ambr250™ system compared to 2 L bioreactors). By looking at the oxygen amount in the off-gas and process oxygen uptake rate (OUR) the “Aged” processes, for example for “48 h” and “96 h”, seem to be more “effective” in oxygen utilization (FIG. 27).

1. A method for treatment of a cell culture medium, the method comprising: holding a cell culture medium in a vessel under conditions suitable for inoculation of the medium with a eukaryotic cell that has been engineered to recombinantly express an exogenous protein, characterized in that the medium comprises the following nutrients
one or more of cystine, cysteine and a cysteine derivative; and
one or more redox active trace metals
and in that the medium comprising the nutrients is held under said conditions suitable for inoculation for a period of at least 10 hours in the absence of cells prior to inoculation.

2. The method according to claim 1 wherein the medium is supplemented with both of (1) one or more of cystine, cysteine and a cysteine derivative; and (2) a redox active trace metal:
(i) before addition of the medium to the vessel;
(ii) at the same time as addition of the medium to the vessel;
(iii) after addition of the medium to the vessel and before the vessel is under conditions suitable for inoculation of the medium with the eukaryotic cell; or
(iv) in the vessel, when the vessel is under conditions suitable for inoculation of the medium with the eukaryotic cell.

3. The method according to claim 1 wherein the medium is held under the conditions suitable for inoculation for a period of at least about 24 hours, at least about 48 hours, at least about 72 hours or at least about 96 hours.

4. The method according to claim 2,
wherein said supplementation takes place before addition of the medium to the vessel.

5. The method according to claim 2, wherein said supplementation takes place after addition of the medium to the vessel.

6. The method according to claim 1, wherein the vessel is a bioreactor.

7. The method according to claim 2, wherein the supplemented medium is in a bioreactor at or prior to inoculation thereof.

8. The method according to claim 1 wherein the cysteine derivative is selected from the group consisting of: S-sulfocysteine, S-sulfocysteinylglycine, N-acetyl cysteine (NAC), cysteine S-linked N-acetyl glucosamine (GlcNAC-cys), homocysteine, L-cysteine mixed disulphides or various peptides, S-alkylated cysteine with a thiol-protecting group, cysteine with a thiol-protecting group, reduced glutathione (GSH), oxidized GSH, S-sulfoglutathione, γ-glutamylcysteine, cysteinylglycine, N-butanoyl α-glutamyl-cysteinyl-glycine, S-acyl-GSH and S-carboxy-L-cysteine.

9. The method according to claim 1 wherein the medium comprises about 0.5 mM to about 16 mM of one or more of cystine, cysteine and a cysteine derivative for at least about 10 hours at conditions suitable for inoculation.

10. The method according to claim 1, wherein the redox active trace metal is one or more selected from the group consisting of iron, copper, chromium, cobalt, selenium, manganese, vanadium and molybdenum.

11. The method according to claim 10, wherein the one or more of iron, copper, chromium, cobalt, selenium, manganese, vanadium and molybdenum is added to the medium as a Trace Element Solution at a cumulative concentration of about 6 μM to about 350 μM.

12. The method according to claim 1 wherein conditions suitable for inoculation are: a pH of about pH 6.0 to about pH 8.0, a temperature of about 20° C. to about 39° C.; and a % O₂ of about 10 to about 80%.

13. The method according to claim 1 wherein the eukaryotic cell is a mammalian cell, a yeast cell or an insect cell.

14. A cell culture medium obtainable by the method of claim 1.

15. Use of the medium of claim 14 in a eukaryotic cell fermentation process for production of recombinant protein.

16. A method for producing a recombinant protein by recombinant expression from a eukaryotic cell, the method comprising:
subjecting a cell culture medium suitable for cultivating said eukaryotic cell to the method according to claim 1,
inoculating the treated cell culture medium with a eukaryotic cell engineered to recombinantly express an exogenous protein to form a cell culture,
cultivating the cell culture so that the recombinant protein is produced.

17. The method of claim 16, wherein the recombinant protein is an antibody.

18. The method according to claim 16, further comprising a step of recovering the recombinant protein from the cell culture.
